(19) United States
(12) Patent Application Publication
(10) Pub. No.: US 2013/0172705 A1
(43) Pub. Date: Jul. 4, 2013

(54) ENZYME CO-FACTOR ENHANCEMENT OF BIOSENSOR PERFORMANCE

(71) Applicants: Peter A. Petillo, Lawrence, KS (US); Daniel V. Aillon, Lawrence, KS (US); Brian S. Barrett, Lawrence, KS (US)

(72) Inventors: Peter A. Petillo, Lawrence, KS (US); Daniel V. Aillon, Lawrence, KS (US); Brian S. Barrett, Lawrence, KS (US)

(73) Assignee: PINNACLE TECHNOLOGY, INC., Lawrence, KS (US)

(21) Appl. No.: 13/821,903

(22) PCT Filed: Sep. 12, 2011
(86) PCT No.: PCT/US11/51193

§ 371 Date: Mar. 8, 2013

Related U.S. Application Data
(60) Provisional application No. 61/381,695, filed on Sep. 10, 2010.

(51) Int. Cl. C12N 9/96; A61B 5/145; A61B 5/1486

(52) U.S. Cl. CPC C12N 9/96; A61B 5/14865; A61B 5/14532
USPC 600/347; 435/174; 435/179; 435/177; 435/180; 204/403.14; 600/345

(57) ABSTRACT

The present invention is broadly concerned with the crafting and manufacturability of an implantable enzymatic-based sensor characterized by a small size, optimum geometry, linearity of response over the concentration range of interest, extended shelf-life, selectivity for the analyte in question, and the ability to exclude bioactive interferents. More particularly, it is preferably concerned with a general approach to optimize the performance of the biorecognition elements required to produce biosensors of the type designed to provide, and in conjunction with a suitable signal processing unit, a current which is proportional to the concentration of the analyte of interest. The biosensors described herein may be implanted in vivo, including intra-cerebral, sub-cutaneous, intra-muscular, inter-peritoneal oral, serum, and vascular implantation, the majority of which may act as a surrogate for systemic monitoring and used to monitor analytes of interest in real-time. Multiple biosensors can be joined together to allow for the simultaneous recording of multiple analytes of interest. In addition to the in vivo applications, sensors of the design described herein may also find use in medical monitoring, industrial processes, fermentation, environmental monitoring, and waste water stream monitoring. The present invention offers co-factor enhancement of the biorecognition element, providing access to a range of biorecognition elements heretofore difficult to incorporate into a manufacturing process for the large-scale production of biosensors.

### RELATED APPLICATIONS

This application claims the priority benefit of a provisional application entitled ENZYME CO-FACTOR ENHANCEMENT OF BIOSENSOR PERFORMANCE, U.S. Patent Application No. 61/381,695, filed Sep. 10, 2010, incorporated by reference herein,

### BACKGROUND OF THE INVENTION

### Field of Invention

The present invention is broadly concerned with the crafting and manufacturability of an implantable enzymatic-based sensor characterized by a small size, optimum geometry, linearity of response over the concentration range of interest, extended shelf-life, selectivity for the analyte in question, and the ability to exclude bioactive interferents. More particularly, it is preferably concerned with a general approach to optimize the performance of the biorecognition elements required to produce biosensors of the type designed to provide, and in conjunction with a suitable signal processing unit, a current which is proportional to the concentration of the analyte of interest. The biosensors described herein may be implanted in vivo, including intra-cerebral, sub-cutaneous, intra-muscular, inter-peritoneal, oral, serum, and vascular implantation, the majority of which may act as a surrogate for systemic monitoring and used to monitor analytes of interest in real-time. Multiple biosensors can be joined together to allow for the simultaneous recording of multiple analytes of interest. In addition to the in vivo applications, sensors of the design described herein may also find use in medical monitoring, industrial processes, fermentation, environmental monitoring, and waste water stream monitoring. The present invention offers co-factor enhancement of the biorecognition element, providing access to a range of biorecognition elements heretofore difficult to incorporate into a manufacturing process for the large-scale production of biosensors.

### BRIEF DESCRIPTION OF THE DRAWING FIGURES

FIG. 1. Sensitivity/activity response of lactate biosensors to a single dose of 0.1 mM solution of sodium L-lactate analyte, where the biosensors were fabricated from lactate oxidase incubated with different co-factors pre-immobilization.

FIG. 2. Sensitivity/activity response of lactate biosensors to a single dose of 0.1 mM solution of sodium L-lactate analyte, where the biosensors were fabricated from apo-lactate oxidase incubated with different co-factors pre- and post-immobilization.

FIG. 3. Limited rescue of a lactate biosensor was shown by its response to a single dose of 0.1 mM solution of sodium L-lactate analyte pre-rescue, immediately post-rescue and two days post-rescue.

FIG. 4. Sensitivity/activity dose-response of lactate biosensors to the addition of sodium L-lactate wherein the lactate oxidase was incubated with FAD co-factor prior to immobilization and tested at (a) 24 hours after preparation, (b) 1 week after preparation, (c) 2 weeks after preparation, (d) 3 weeks after preparation, and (e) 4 weeks after preparation.

FIG. 5. Sensitivity/activity dose-response of lactate biosensors to the addition of sodium L-lactate wherein the lactate oxidase was incubated with FAD co-factor prior to immobilization and wherein the linearity of the response to increasing amounts of lactate was revealed.

FIG. 6. In vivo response of lactate and glucose biosensors over a 24 hour period, wherein the lactate oxidase was incubated with FAD co-factor prior to immobilization, and the lactate and glucose biosensors were coated with a polyurethane layer prior to implantation.

FIG. 7. Extended in vivo response of lactate and glucose biosensors over a seven day period, wherein the lactate oxidase was incubated with FAD co-factor prior to enzyme immobilization, and the lactate and glucose biosensors were coated with a polyurethane layer prior to implantation.

FIG. 8. Interference plot of a lactate biosensor against a range of common electroactive interferents, wherein the lactate oxidase was incubated with FAD co-factor prior to enzyme immobilization.

### SUMMARY OF THE INVENTION

The present invention broadly provides a method of forming a sensor, where the method comprises incubating an enzyme in the presence of a co-factor for the enzyme, and immobilizing the enzyme in its active form.

In another embodiment, a sensing element is provided. The sensing element comprises a support having a surface. The element further comprises a layer on the surface, where the layer comprises an enzyme and a co-factor for the enzyme in a matrix. The enzyme is predominantly in its active form and the matrix retains the enzyme in the active form.

In a further embodiment, the invention is concerned with an amperometric biosensor comprising a working electrode and a reference electrode. The working electrode comprises a sensing element comprising a support having a surface, and a layer on the surface. The layer comprises an enzyme and a co-factor for the enzyme in a matrix, with the enzyme being predominantly in its active form and the matrix retaining the enzyme in the active form.

In yet a further embodiment, an assembly for oral, intra-cerebral, intramuscular, intravascular, vascular, inter-peritoneal, or sub-cutaneous placement and anchoring is provided. The assembly comprises an amperometric biosensor and a device selected from the group consisting of a cannula, a cannula headpiece, a patch, an implant, and a trocar, with the biosensor being attached to the device. The amperometric biosensor comprises:
a working electrode comprising a sensing element comprising:
   a support having a surface; and
   a layer on the surface, the layer comprising an enzyme and a co-factor for the enzyme in
   a matrix, the enzyme being predominantly in its active form and the matrix retaining the enzyme in the active form; and
a reference electrode.

### DETAILED DESCRIPTION OF THE PREFERRED EMBODIMENTS

This invention provides a general approach to yield properly conditioned biorecognition elements that provide the foundation for the construction of biosensors. By definition, biosensors require a biorecognition element to provide activity, specificity, and selectivity to the analyte(s) of interest. The conditioning of a biorecognition element as a discreet step in the fabrication of a properly functioning biosensor is of critical importance and has not been widely recognized or generalized. The biosensors disclosed herein, including those for in vivo biological applications, enjoy a wide range of use, and may be deployed via intra-cerebral, sub-cutaneous, intra-muscular, inter-peritoneal, oral, serum, and vascular implantation, the majority of which may act as a surrogate for systemic monitoring. Applications for biosensors are broad and include monitoring of disease states and infectious diseases (e.g., West Nile Virus and SARS), pathogens e.g., listeria and salmonella), various E. coli contaminations, analytes for drug control and interdiction (e.g., alcohol, cannabis and THC), physiological states (e.g., pregnancy), cholesterol levels, and heart health (e.g. cardiac biomarkers, coagulation PT, coagulation ACT). Biosensors may provide timely data needed to treat trauma and traumatic brain injuries and battlefield insults. A single biosensor may be fabricated for the sensing of multiple analytes of interest. Multiple biosensors can be joined together to allow for the simultaneous recording of multiple analytes of interest. In addition to the in vivo applications, sensors of the design described herein may also find use in medical monitoring, industrial processes, fermentation, environmental monitoring, and waste water stream monitoring. These biosensors are possible because of the conditioning of the biorecognition element prior to its introduction into the sensing matrix.

### Enzymes and Co-Factors

All enzymes are identified and classified according to the Enzyme Commission number (EC number) classification system (Webb, Edwin C., 1992, Enzyme nomenclature 1992: Recommendations of the Nomenclature Committee of the International Union of Biochemistry and Molecular Biology on the nomenclature and classification of enzymes. San Diego: Published for the International Union of Biochemistry and Molecular Biology by Academic Press). The EC system is a numerical classification scheme for enzymes, based on the chemical reaction that is catalyzed. As a system of enzyme nomenclature, every EC number is associated with a recommended name for each enzyme.

Some enzymes do not need any additional components to show full activity. If an enzyme requires one or more non-protein molecules to be bound to the enzyme for activity, these molecules are collectively referred to as co-factors (de Bolster, M. W. G., 1997, “Glossary of Terms Used in Bioinorganic Chemistry: Cofactor”, International Union of Pure and Applied Chemistry). These non-covalently bound molecules are usually found proximal to or part of the active site of the enzyme and are involved in catalysis. For example, flavin and heme co-factors are often involved in redox reactions and the former is often found in oxidoreductases.

Co-factors can be either inorganic compounds (e.g., iron-sulfur clusters and metal ions selected from the group consisting of iron, zinc, selenium, cobalt, magnesium, molybdenum, vanadium, manganese, copper, tungsten, cadmium, and nickel) or organic compounds (e.g., heme, flavin, flavin adenine dinucleotide—FAD, flavin mononucleotide—FMN). Organic co-factors can be either prosthetic groups, which are non-covalently bound to an enzyme, or co-enzymes, which are released from the enzyme's active site during the reaction. Co-enzymes include NAHD, NADPH, and adenosine triphosphate—ATP. The term co-enzyme refers specifically to enzymes and, as such, to the functional properties of a protein. Most co-factors are not covalently attached to an enzyme. In some instances, organic prosthetic groups such as FAD and FMN can be covalently bound.

The term prosthetic group emphasizes the nature of the binding of a co-factor to a protein (tight or covalent) and, thus, refers to a structural property. Different sources give slightly different definitions of co-enzymes, co-factors, and prosthetic groups. Some consider non-covalently bound organic molecules as prosthetic groups and not as co-enzymes, while others define all non-protein organic molecules needed for enzyme activity as co-enzymes and classify those that are non-covalently bound as co-enzyme prosthetic groups. It should be noted that these terms are often used loosely. As used here, the terms “co-factor,” “prosthetic group,” and “co-enzyme prosthetic group” refer to a molecule that is not a protein, that is needed for enzyme activity, and that is non-covalently bound to the enzyme of interest.

The co-factors are not limited to those only found in human iso-forms of a particular enzyme, but also embrace all iso-forms of said enzyme found in all organisms. It is well accepted and understood that different iso-forms of the same enzyme from different organisms will possess different amino acid sequences and may rely on different co-factors for activity. For example, the human iso-form of diamine oxidase (EC 1.4.3.22), sometimes referred to as histamine oxidase, does not require a co-factor for activity. Instead, human diamine oxidase relies on a post-translational hydroxylation of an active site tyrosine residue to a trihydroxyphenylalanine residue and then finally to a topaquinone (TPQ) residue (McGrath, A. P. et al. Biochemistry, 2009, 48(41), 9810-9822). By contrast, the diamine oxidase found in rice seedlings from the species Oryza sativa requires FAD as a co-factor for activity (Chaudhuri, M. M.; Ghosh, B. Phytochemistry, 1984, 23, 241-243).

Enzymes that require a co-factor but do not have one bound are called apo-enzymes or apo-proteins. An apo-enzyme together with its co-factor(s) is called a holo-enzyme or holo-protein. By definition, the holo-enzyme is the competent, active form of the enzyme and is capable of supporting catalysis without the need for other co-factors. The term holo-enzyme can also be applied to enzymes that contain multiple protein subunits, where this collection of subunits is also in a competent active form capable of function.

An example of an enzyme that requires a co-factor for activity is lactate oxidase (Streitenberger, S. A.; et al. J. Enzyme Inhib. Med. Chem. 2003, 18, 285-288), which can use either FAD or FMN to form the holo-enzyme and promote catalysis. In the absence of either co-factor, the apo-form of the enzyme is catalytically inactive and cannot promote catalysis.

### Co-Factor Enhancement of Biosensor Biorecognition Element(s)

The construction of biosensors has been reviewed (Wilson, G. S.; Johnson, M. A.; Chem Rev., 2008, 108(7), 2462-81. Johnson, D. A.; Wilson, G. S.; In: Michael A C, Borland L M, editors. Electrochemical Methods for Neuroscience. Boca Raton (FL): CRC Press; 2007. Chapter 20. Wilson, G. S.; Gifford, R.; Biosens Bioelectron., 2005, 20(12), 2388-403. Matsumoto, N.; Chen, X.; Wilson G. S.; Anal Chem., 2002, 74(2), 362-7. Ward W. K.; Jansen, L. B.; Anderson, E.; Reach, G.; Klein, J. C.; Wilson, G. S.; Biosens Bioelectron., 2002, 17(3), 181-9). In general, biosensors are composed of four components: (1) a sensing electrode (the transducing element or component) typically fashioned from platinum, a platinum/iridium alloy, gold, silver, other precious metals, alloys or glasses, and where said sensing element can support an electrochemical or optical measurement; (2) an inner-membrane that provides a separation layer between the sensing electrode and the enzyme layer and which helps to enhance the selectivity of the biosensor; (3) an enzyme layer or sensing matrix that is minimally composed of one or more biorecognition element(s) and which may also have other proteins and non-protein molecules as part of its composition; and (4) an outer-membrane layer which may help regulate the flux of the analyte of interest to the other layers. For the purposes of this invention, the sensing layer (1), the inner-membrane (2) and the outer-membrane (4) can be considered generic layers in that this invention will be equally applicable to all sensing layers, all inner-membranes, and all outer-membranes capable of supporting biosensor construction. These layers do not necessarily need to be present as discreet distinct layers and may in fact be intermingled. One or more of the generic layers may be absent in the final biosensor design and fabrication. However, if an enzyme layer or sensing matrix exists as part of a biosensor, and said enzyme layer has a co-factor dependent enzyme as part of said layer, then this invention will find utility.

By definition, the term biosensor specifically refers to a sensor that employs a biorecognition element to provide analyte recognition and analyte specificity (Wilson G. S.; Ammam, M. FEBS J. 2007, 274(21), 5452-61). The biorecognition element(s) used as part of the biosensor construction are typically oxidoreductases, which are a sub-class of flavoproteins (Macheroux, P. et al. FEBS J. 2011, 278, 2625-2634). Oxidoreductases often catalyze the conversion of a substrate with the generation of electroactive hydrogen peroxide as a by-product. The amperometric measure of the hydrogen peroxide generated thus provides the means by which the presence of the substrate analyte is determined. In principle, any oxidoreductase that produces an electroactive by-product and which requires a co-factor(s) for activity can be utilized as part of this invention.

By definition, the term sensitivity used herein refers to the electrical response as a function of concentration and refers to the biosensor composite. Activity refers to a measure of the performance of an enzyme (biorecognition element), which may be co-factor driven, and is typically defined in terms of units, wherein one unit is defined as the amount of enzyme which generates 1 μmole of H₂O₂ per minute at 37° C. under a set of standard conditions, and where standard condition may vary from enzyme to enzyme.

Flavoproteins are proteins that contain a nucleic acid derivative of riboflavin: tFAD or FMN (Dym, O.; Eisenberg, D.; Protein Sci., 2001, 10(9), 1712-1728). Flavoproteins are involved in a wide array of biological processes, including, but by no means limited to, bioluminescence, removal of radicals contributing to oxidative stress, photosynthesis, DNA repair, and apoptosis flavoproteins (Macheroux, P. et al. FEBS J. 2011, 278, 2625-2634). The spectroscopic properties of the flavin co-factor make it a natural reporter for changes occurring within the active site; this makes flavoproteins one of the most-studied enzyme families (Massey, V.; Biochem. Soc. Trans. 2000, 28, 283-296). The first evidence for the requirement of flavin as an enzyme co-factor came in 1935 (Theorell, H.; Biochemische Zeitschrift 1935, 275, 344-346). Hugo Theorell and coworkers showed that a bright-yellow-colored yeast protein, identified previously as essential for cellular respiration, could be separated into apoprotein and a bright-yellow pigment. Neither apo-protein nor pigment alone could catalyse the oxidation of NADH, but mixing of the two restored the enzyme activity. However, replacing the isolated pigment with riboflavin did not restore enzyme activity, despite their being indistinguishable under spectroscopy. This led to the discovery that the protein studied required not riboflavin but FMN to be catalytically active.

Oxidoreductases generally require FAD or FMN as co-factors, but are not limited to just these groups. Some members of the family are known to require one or more of the following co-factors for activity: thiamine pyrophosphate (TPP), quinone, heme, copper, and other heavy metals ions selected from the group consisting of iron, zinc, selenium, cobalt, magnesium, molybdenum, vanadium, manganese, copper, tungsten, cadmium, and nickel. Some family members rely upon post-translation modification(s) of active site amino acid residues, which results in a covalently modified protein where the co-factor(s) is an integral part of the enzyme. Enzyme preparations that do not include the addition of the co-factor(s) universally result in loss of enzymatic activity, which may be restored by the addition of co-factor(s) in solution. The flavoprotein family displays a wide range of activities and stabilities, with loss of activity directly linked to loss of co-factor(s). As a general rule, the co-factors are not covalently bound, although there are exceptions such as glucose oxidase (EC 1.1.3.4). Temperature plays an important role in the loss of co-factor, as higher temperatures lead to faster loss of co-factor. Considerable effort has been described in developing temperature stable mutants of flavoproteins, most especially for those flavoproteins with direct applications in human analyte monitoring (e.g., glucose, glutamate, alcohol, lactate, histamine, etc). While modern molecular biology techniques allow for the directed evolution of temperature stable flavoprotein mutants, such mutants can often affect the fidelity of the catalytic process and lead to a protein that is less active than the wild-type.

Enzyme preparations, produced via recombinant methods or obtained by isolation from natural sources, may contain high proportions of apo-protein. The functional stability of flavoproteins is known to directly relate to an enzyme's ability to retain co-factor(s). Restoration of the activity by the addition of co-factor(s) will likely reflect conditions that help drive the equilibrium of the system to produce the fully activated form of the enzyme (based on Le Chatelier's principle). Factors that can positively contribute to the restoration of enzyme activity include the co-factor to enzyme ratio, incubation temperature of the co-factor/enzyme mixture, and the incubation time of said mixture. The ratio of co-factor(s) to enzyme can range from about 1:1 to about 100,000,000:1 or larger, preferably from about 1:1 to about 100,000:1, and more preferably from about 1:1 to about 10,000:1. The incubation temperature can range from about −10° C. to about 100° C., preferably from about −10° C. to about 50° C., and more preferably from about -10 ° C. to about 25° C. The incubation time can range from about zero seconds (immediate use) to about 72 hours, preferably from about 1 minute to about 12 hours, and more preferably from about 1 minute to about 3 hours. A good set of starting conditions is to provide a 10,000:1 ratio of co-factor(s) to enzyme and to allow the mixture to incubate at 0° C. for 90 minutes. The optimal ratio of co-factor to enzyme, the length of incubation and the temperature of incubation is enzyme dependent and may be determined and optimized for each enzyme to be employed as biorecognition elements. Further, if multiple co-factors are required for activity, the order of addition of the co-factors may be important.

Enzyme-based biorecognition elements may benefit from the incubation with co-factor(s) prior to immobilization in a solid matrix. Incubation of the biorecognition element with known co-factor(s) enhances the activity of the enzyme and coverts all apo-protein to active, fully functional holo-protein (the limiting case). This fulfills the catalytic potential for activity of the mass of the enzyme in the sensing matrix used in the biosensor construction. The importance of incubation of the biorecognition element with co-factors prior to immobilization has not been generally recognized and is key to the manufacturability of biosensors, most especially those biosensors whose biorecognition element is co-factor driven and whose overall stability as a function of time and temperature is not robust. The oxidoreductase enzymes in Table 1 benefit from conditioning prior to introduction of the enzyme, as the biorecognition element, into the enzyme layer of each biosensor. The following oxidoreductases dramatically benefit from incubation: Lactate oxidase (EC 1.1.3.15), D-amino acid oxidase (EC 1.4.3.3), (S)-6-Hydroxynicotine oxidase (EC 1.5.3.5), (R)-6-Hydroxynicotine oxidase (EC 1.5.3.6), Alcohol oxidase (EC 1.1.3.13), Pyruvate oxidase(EC 1.2.3.3), Glucose oxidase (EC 1.1.3.4), Glutamate oxidase (EC 1.4.3.11), Acyl coenzyme A oxidase (EC 1.3.3.6), Choline oxidase (EC 1.1.3.17), Glutathione Sulfhydryl oxidase (EC 1.8.3.3), Glycerolphosphate oxidase (EC 1.1.3.21), Sarcosine oxidase (EC 1.5.3.1), Xanthine oxidase (EC 1.1.3.22), Oxalate oxidase (EC 1.2.3.4), Cholesterol oxidase (EC 1.1.3.6), Gamma-glutamyl-putrescine oxidase (EC undefined), GABA oxidase (EC undefined), Histamine oxidase (Diamine oxidase, EC 1.4.3.22), Nucleoside oxidase (EC 1.1.3.39), L-Lysine oxidase (EC 1.4.3.14), L-Aspartate oxidase (EC 1.4.3.16), Glycine oxidase (EC 1.4.3.19), and Galactose oxidase (EC 1.1.3.9). GABA is defined as gamma alpha-butyric acid.

Biosensor response (also referred to as the biosensor performance and biosensor sensitivity) is monitored in vitro by testing the sensor against a known concentration of the analyte of interest in a buffered solution. When the analyte of interest comes into contact with the enzyme layer of the biosensor, the enzyme catalytically processes the analyte to produce H₂O₂ as a by-product. The amount of H₂O₂ produced is directly proportional to the concentration of the analyte. The response of the biosensor results from the oxidation, at the transducing element, of the enzymatically produced H₂O₂ and is recorded as a current, commonly referred to as an oxidation current. The oxidation current is typically reported in nanoamps, a convention that will be preserved herein.

Oxidoreductases possess a range of stabilities, which usually differ between the apo- and holo-forms of each enzyme. Key stability parameters that are usually not experimentally determined are the kinetics and thermodynamics of co-factor exchange. Unless the co-factor(s) is covalently attached to the oxidoreductase, said co-factor(s) may be lost from the enzyme as a function of time and temperature. Most preparations of oxidoreductases exist as a mixture of apo- and holo-enzyme. Lactate oxidase (EC 1.1.3.15 or EC 1.13.12.10) is one of the more robust oxidase enzymes, and preparations of the purified enzyme often achieve activities that are suitable for direct use.

### EXAMPLES 1, 2, 3, 4 AND 5

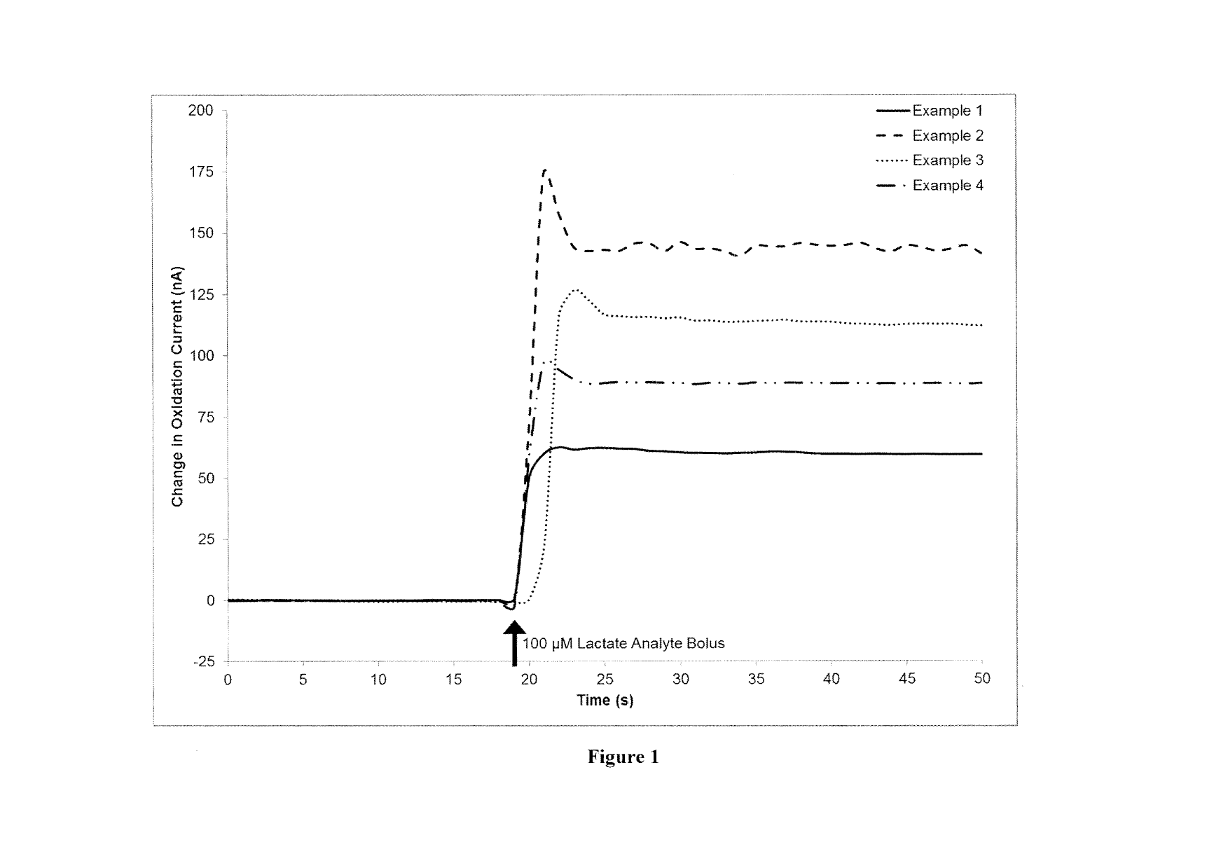

The biosensor in Example 1 (Table 1) showed the response to lactate analyte where said biosensor was fabricated from a typical commercial preparation of lactate oxidase. Even though biosensor response was observed, the overall sensitivity of the biosensor to lactate analyte could be significantly increased by pre-incubating, prior to immobilization, the lactate oxidase used to fabricate the biosensor. Co-factors FMN co-(Example 2, the natural co-factor), FAD (Example 3), and riboflavin (Example 4) all result in improved biosensor response to lactate analyte when the co-factors are individually incubated with the lactate oxidase enzyme prior to immobilization into the sensing cavity of the biosensor. This is shown in FIG. 1 where the responses of the biosensors in Examples 1-4 were plotted on the same graph and the positive effect of the pre-incubation of the lactate oxidase with co-factor prior to immobilization was clearly discernible. This improvement in biosensor response only occurred when the enzyme was pre-incubated with the appropriate co-factor(s) prior to immobilization into the enzyme matrix and is contrary to the accepted practice of reactivating an oxidoreductase at any point in time. This is further demonstrated in Example 5, which is the biosensor from Example 1 incubated with FMN after placement in the enzyme matrix. No discernible difference is observed, indicating that the FMN and FAD incubation must occur prior to placement of the enzyme into the enzyme layer. Prior to the application of a generic outer-membrane, the biosensors in Examples 2, 3, and 4 showed a linear response range (>95% linear) up to 400 μM of lactate analyte. In the presence of a generic outer-membrane, this linear response range (>95% linear) improved to over 5 mM of lactate analyte.

### EXAMPLES 6, 7, 8, AND 9

The biosensors in Examples 6, 7, and 8 further demonstrated and supported this concept, where apo-lactate oxidase was made into a biosensor without the benefit of pre-incubation with co-factor prior to immobilization. Consistent with this invention, the biosensor fabricated with no pre-incubation of the enzyme with co-factor prior to immobilization failed to respond to a lactate analyte bolus (Example 6). If such a biosensor from Example 6 was allowed to incubate with FMN (Example 7) or FAD (Example 8), at either 25° C. or 37° C., rescue of the enzyme activity and therefore the biosensor response did not occur and the biosensor was still unable to respond to a lactate analyte bolus to any appreciable extent.

Rescue of a biosensor response is specifically defined as the post-immobilization incubation of said finished biosensor in a concentrated solution of the appropriate co-factor(s) at either 25° C. or 37° C. for at least 30 minutes to reactivate the enzyme prior to measurement of the biosensor's response. Note that these conditions are typically suitable for the reactivation of an oxidoreductase in solution.

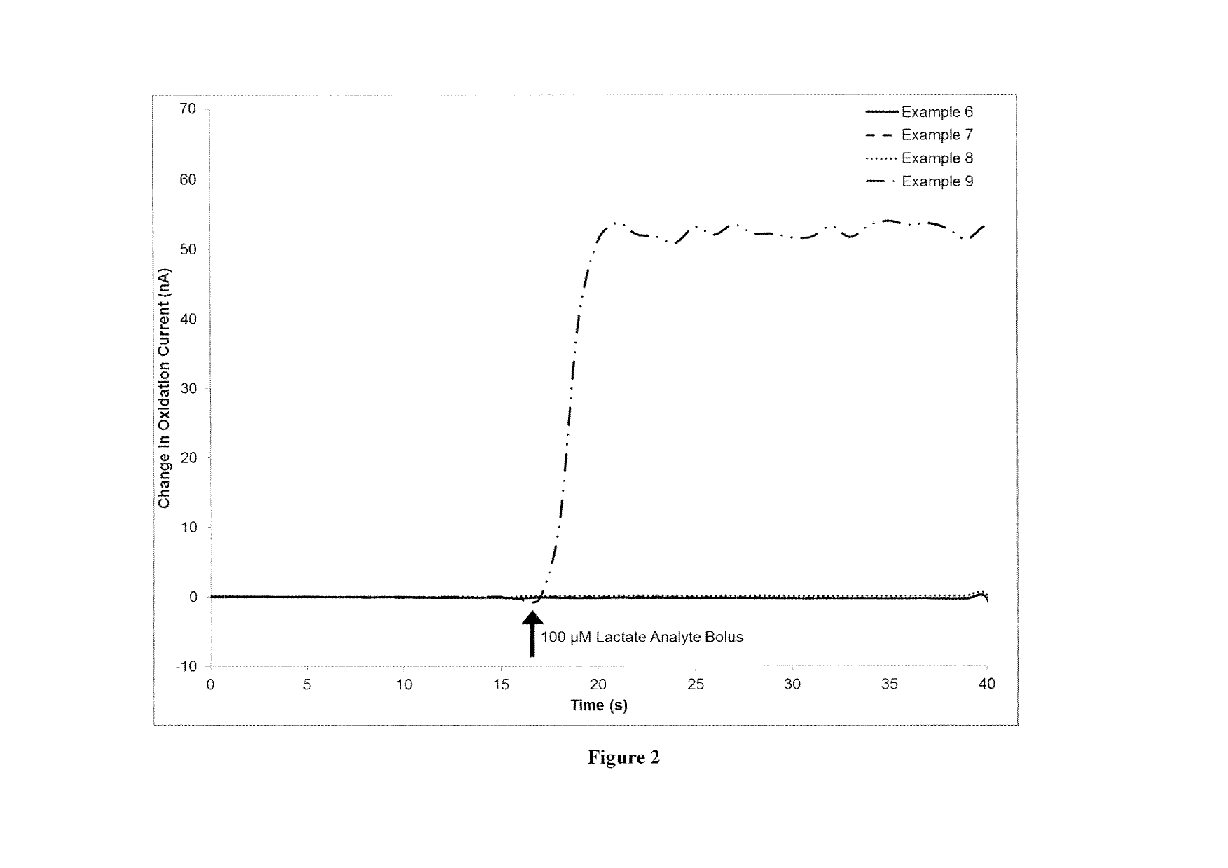

The order of addition of the co-factor is of critical importance for the purposes of fashioning a functioning biosensor, and this invention has not been articulated nor would it be obvious to one skilled in the art. The apo-lactate oxidase that gave no discernible signal in the absence of co-factor pre-incubation prior to immobilization (when integrated into a biosensor, Examples 6, 7, and 8) was converted into a competent enzyme suitable for biosensor construction by pre-incubating the apo-lactate oxidase with FMN co-factor prior to immobilization as shown in Example 9. This was also shown in FIG. 2 where Examples 6-9 are plotted on the same graph and the positive effect of the pre-incubation of co-factor prior to immobilization was clearly discernible. The difference in absolute activities between the enzyme used for Examples 1-5 and that used for Examples 6-9, when fully activated, reflects the batch-to-batch differences that can be observed. Batch-to-batch variations do occur, and this invention provides a rational approach for insuring that every batch of enzyme is fully activated when utilized for biosensor construction regardless of the starting apo-to-holo ratio.

### EXAMPLES 10, 11 AND 12

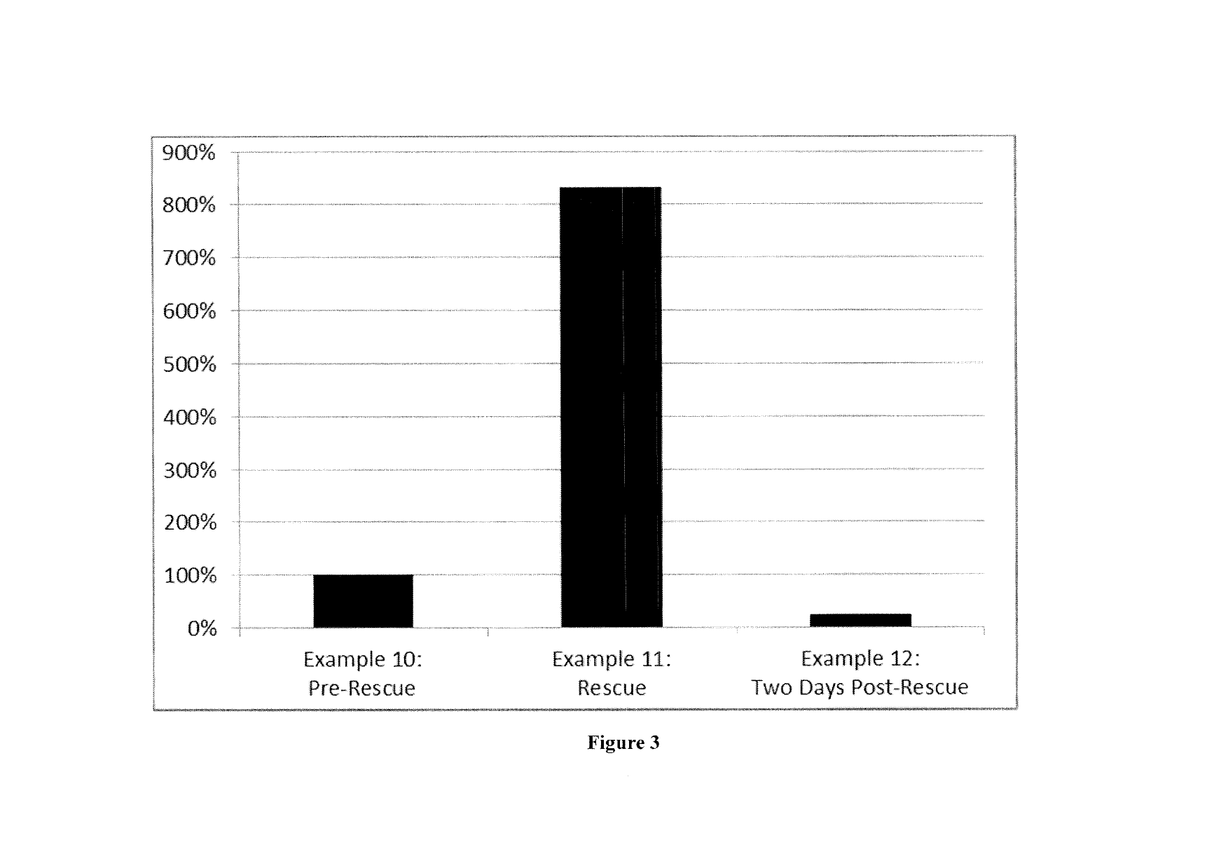

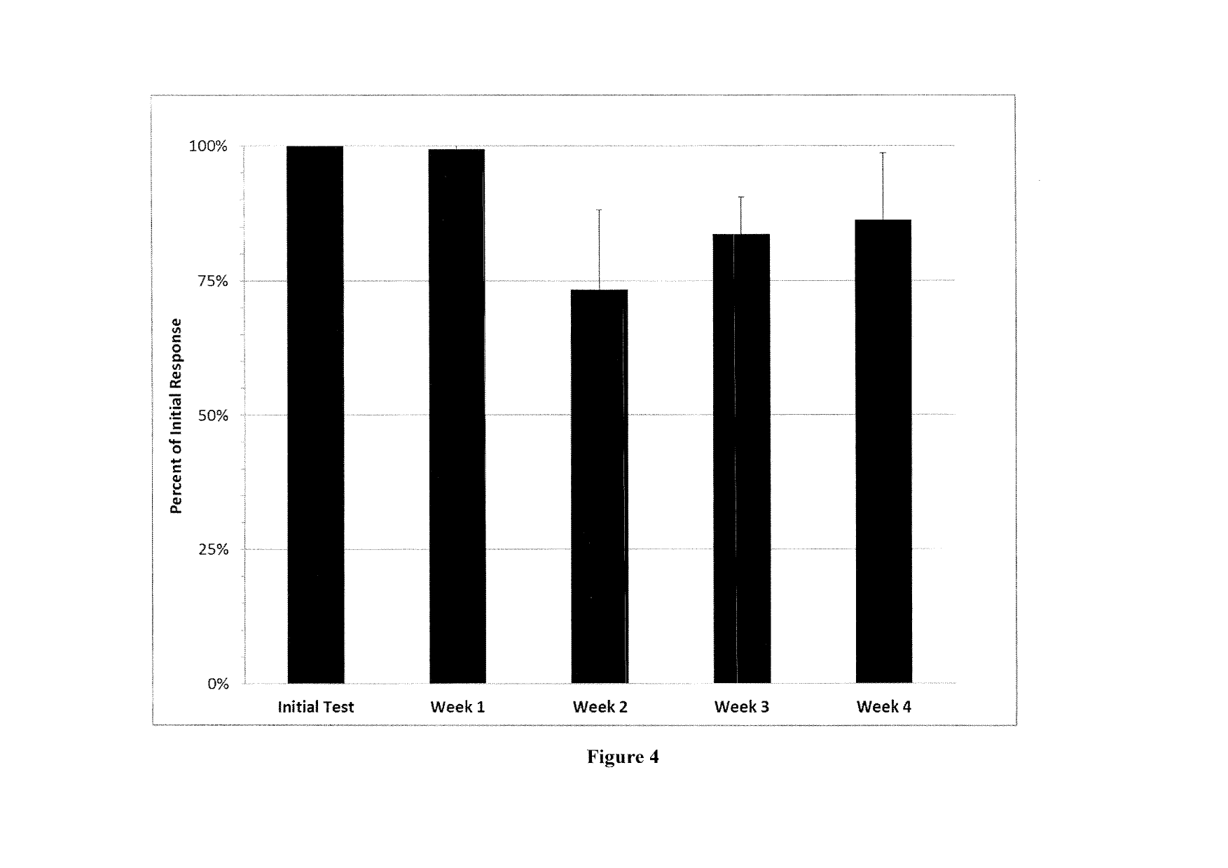

The biosensors in Examples 10-12 and FIGS. 3 and 4, revealed that a limited rescue was observed of a lactate biosensor fabricated from lactate oxidase enzyme that was not pre-incubated with co-factor prior to immobilization. The biosensor response in Example 10 showed a dramatic increase in response to lactate analyte upon FAD incubation (Example 11), but this increase was short-lived and within two days, the biosensor response dropped below pre-rescue levels (Example 12). This is shown in FIG. 3 where Examples 10-12 are plotted on the same graph and the short-lived effect of the rescue is clearly discernible. By contrast, lactate biosensors fabricated from lactate oxidase enzyme pre-incubated with FAD or FMN co-factor prior to immobilization retain their activity to lactate analyte for over a month and to such a degree that rescue was not needed (FIG. 4),

The difference in biosensor response (to a lactate analyte bolus) for biosensors fabricated from the same apo-lactate oxidase enzyme that was pre-incubated with co-factor prior to immobilization (Example 9) versus those biosensors that were incubated with co-factor only after enzyme immobilization (Examples 7 and 8) was profound and indicative of the importance of this invention (FIG. 2). Furthermore, this effect was not solely due to the enzyme itself, but reflected the importance of crafting an enzyme layer suitable for use in a biosensor. The final matrix into which the enzyme became embedded could only be properly formed when the enzyme was pre-incubated in the presence of the appropriate co-factor(s) before said matrix was formed (Examples 2, 3, 4, and 9). Rescue of the enzyme was possible (Example 10 versus Example 11), but this rescue was short-lived (Example 11 versus Example 12) and was not compatible with the properties of a functioning biosensor, especially for in vivo use. Furthermore, an enzyme that was not pre-incubated with co-factor prior to immobilization (Example 10), resulted in a enzyme matrix predisposed to the rapid loss of co-factor (Example 12), a situation which was readily prevented by by pre-incubation of the enzyme with co-factor(s) prior to immobilization (Examples 2, 3, 4, and 9).

### EXAMPLES 13, 14, AND 15

Examples 13, 14, and 15 showed biosensors fabricated from D-amino acid oxidase enzyme. Functional biosensors (as determined by the biosensor response to D-serine analyte) were best achieved when said biosensors were fabricated from D-amino acid oxidase enzyme that was pre-incubated with FAD co-factor prior to immobilization into the enzyme matrix. The enzyme used to fabricate the biosensor in Example 13 was pre-incubated with FAD prior to immobilization and said biosensor responded to D-serine analyte. If rescue (Example 15) of a biosensor's response was attempted by co-factor incubation after the enzyme was immobilized in the matrix but without conditioning (by pre-incubation with co-factor prior to immobilization, Example 14), the biosensor response was not restored versus that seen for Example 13. Prior to the application of a generic outer-membrane, the biosensor in Example 13 had a linear response range (>95% linear) of over 40 μM of D-serine analyte.

### EXAMPLES 16 AND 17

The biosensor in Example 16 was fabricated from D-amino acid oxidase enzyme where the activity of the D-amino acid oxidase enzyme and the function of the biosensor were enabled by pre-incubating the enzyme with FAD co-factor prior to immobilization. At least 73% of the biosensor response was preserved after four months (Example 17) relative to the starting biosensor response (Example 16).

### EXAMPLES 18, 19, 20, AND 21

Examples 18, 19, 20, and 21 showed biosensors fabricated from L-6-hydroxynicotine oxidase enzyme. Functional biosensors (as determined by the biosensor response to analyte) were best achieved when said biosensors were fabricated from L-6-hydroxynicotine oxidase enzyme that was pre-incubated with FAD co-factor prior to immobilization into the enzyme matrix. These biosensors responded to nicotine analyte (Example 18), anabasine analyte (Example 20) and nor-nicotine analyte (Example 21). If a biosensor was fabricated with L-6-hydroxynicotine oxidase enzyme that was not conditioned by pre-incubation with FAD co-factor prior to immobilization (Example 19), said biosensor failed to respond to nicotine analyte when tested at the same concentration as Example 18. Prior to the application of a generic outer-membrane, the biosensors in Example 18, 20 and 21 had linear response ranges (>95% linear) of over 60 μM of nicotine, anabasine and nor-nicotine analytes respectively.

### EXAMPLES 22 AND 23

Examples 22 and 23 showed biosensors fabricated from alcohol oxidase enzyme. Functional biosensors (as determined by the biosensor response to ethanol analyte) were best achieved when said biosensors were fabricated from alcohol oxidase enzyme that was pre-incubated with FAD co-factor prior to immobilization into the enzyme matrix. The biosensor in Example 22 was pre-incubated with FAD prior to immobilization and responded to ethanol analyte. The enzyme used to fabricate the biosensor in Example 23 was not pre-incubated with FAD prior to immobilization and said biosensor failed to respond to ethanol analyte when tested at the same concentration as Example 22. After the application of a generic outer-membrane, biosensors fashioned as Example 22 had a linear response range (>95% linear) of over 50 mM of ethanol analyte in oxygen enriched media.

### EXAMPLES 24-28

Examples 24, 25, 26, 27 and 28 showed biosensors fabricated from pyruvate oxidase enzyme. Functional biosensors (as determined by the biosensor response to pyruvate analyte) were best achieved when said biosensors were fabricated from pyruvate oxidase enzyme that was pre-incubated with both FAD and TPP co-factors prior to immobilization into the enzyme matrix (Example 24). If a biosensor (Example 25) was fabricated from pyruvate oxidase that was not pre-incubated with either co-factor prior to immobilization no response to pyruvate analyte was observed. If a biosensor was fabricated from pyruvate oxidase that was pre-incubated with only FAD co-factor prior to immobilization, no response to pyruvate analyte was observed (Example 26). If a biosensor is fabricated from pyruvate oxidase that was pre-incubated with only TPP co-factor prior to immobilization, no response to pyruvate analyte is observed (Example 27). If a biosensor was fabricated from pyruvate oxidase that was pre-incubated with only FAD co-factor prior to immobilization, no response to pyruvate analyte was observed even if the amount of enzyme used was increased five-fold (Example 28).

Examples 24-28 also demonstrated the utility when more than one co-factor was needed for enzyme activation. Pyruvate oxidase enzyme requires both FAD and TPP to support the catalytic cycle of the enzyme. If both co-factors were present (Example 24), then activity of the biosensor towards pyruvate analyte was observed. If both co-factors were absent (Example 25) or if either FAD was used alone (Example 26) or TPP is used alone (Example 27), then no activity towards pyruvate was observed. An increase in the enzyme concentration five-fold over the standard conditions but with just FAD present (TPP was absent) also resulted in a biosensor that failed to sense pyruvate (Example 28).

### EXAMPLES 29 AND 30

Examples 29 and 30 showed biosensors fabricated from glucose oxidase enzyme. Functional biosensors (as determined by the biosensor response to glucose analyte) were best achieved when said biosensors were fabricated from glucose oxidase enzyme that was pre-incubated with FAD co-factor prior to immobilization into the enzyme matrix. The biosensor in Example 29 was pre-incubated with FAD prior to immobilization and responded best to glucose analyte. The enzyme used to fabricate the biosensor in Example 30 was not pre-incubated with FAD prior to immobilization and the said biosensor showed over a 10-fold reduction in response to glucose analyte when tested at the same concentration as Example 29. Prior to the application of a generic outer-membrane, the biosensor in Examples 29 showed a linear response range (>72% linear) up to 4 mM of glucose analyte. In the presence of a generic outer-membrane, this linear response range (>95% linear) improved to over 5 mM of glucose analyte.

### EXAMPLES 31 AND 32

Examples 31 and 32 showed biosensors fabricated from glutamate oxidase enzyme. Functional biosensors (as determined by the biosensor response to glutamate analyte) were best achieved when said biosensors were fabricated from glutamate oxidase enzyme that was pre-incubated with FAD co-factor prior to immobilization into the enzyme matrix. The biosensor in Example 31 was pre-incubated with FAD prior to immobilization and responded to glutamate analyte. The enzyme used to fabricate the biosensor in Example 32 was not pre-incubated with FAD prior to immobilization and the said biosensor failed to respond to glutamate analyte when tested at the same concentration as Example 31. Prior to the application of a generic outer-membrane, the biosensor in Example 31 had a linear response range (>99% linear) of over 40 μM of glutamate analyte.

### EXAMPLE 33

Example 33 showed a biosensor fabricated from acyl Co-A oxidase enzyme. The functional biosensor (as determined by the biosensor response to N-butyrl Co-A analyte) was best achieved when said biosensor was fabricated from acyl Co-A oxidase enzyme that was pre-incubated with FAD co-factor prior to immobilization into the enzyme matrix. The enzyme used to fabricate the biosensor in Example 33 was pre-incubated with FAD prior to immobilization and said biosensor responded to N-butyrl Co-A analyte. Prior to the application of a generic outer-membrane, the biosensor in Example 31 had a linear response range (>98% linear) of over 40 μM of N-butyrl Co-A analyte.

### EXAMPLE 34

Example 34 showed a biosensor fabricated from choline enzyme. The functional biosensor (as determined by the biosensor response to choline analyte) was best achieved when said biosensor was fabricated from choline oxidase enzyme that was pre-incubated with FAD co-factor prior to immobilization into the enzyme matrix. The enzyme used to fabricate the biosensor in Example 34 was pre-incubated with FAD prior to immobilization and said biosensor responded to choline analyte. Prior to the application of a generic outer-membrane, the biosensor in Example 34 had a linear response range (>99% linear) of over 40 μM of choline analyte.

### EXAMPLE 35

Example 35 showed a biosensor fabricated from glutathione sulfhydryl oxidase enzyme. The functional biosensor (as determined by the biosensor response to reduced glutathione analyte) was best achieved when said biosensor was fabricated from glutathione sulfhydryl oxidase enzyme that was pre-incubated with FAD co-factor prior to immobilization into the enzyme matrix. Prior to the application of a generic outer-membrane, the biosensor in Example 35 had a linear response range (>98% linear) of over 5 mM of reduced glutathione analyte.

### EXAMPLE 36

Example 36 showed a biosensor fabricated from glycerolphosphate oxidase enzyme. The functional biosensor (as determined by the biosensor response to glycerol-3-phosphate analyte) was best achieved when said biosensor was fabricated from glycerolphosphate oxidase enzyme that was pre-incubated with FAD co-factor prior to immobilization into the enzyme matrix. The enzyme used to fabricate the biosensor in Example 36 was pre-incubated with FAD prior to immobilization and said biosensor responded to glycerol-3-phosphate analyte. Prior to the application of a generic outer-membrane, the biosensor in Example 36 had a linear response range (>99% linear) of over 300 μM of glycerol-3-phosphate analyte.

### EXAMPLE 37

Example 37 showed a biosensor fabricated from sarcosine oxidase enzyme. The functional biosensor (as determined by the biosensor response to sarcosine analyte) was best achieved when said biosensor was fabricated from sarcosine oxidase enzyme that was pre-incubated with FAD co-factor prior to immobilization into the enzyme matrix. The enzyme used to fabricate the biosensor in Example 37 was pre-incubated with FAD prior to immobilization and said biosensor responded to sarcosine analyte. Prior to the application of a generic outer-membrane, the biosensor in Example 37 had a linear response range (>98% linear) of over 1 mM of sarcosine analyte.

### EXAMPLE 38

Example 38 showed a biosensor fabricated from xanthine oxidase enzyme. The functional biosensor (as determined by the biosensor response to xanthine analyte) was best achieved when said biosensor was fabricated from xanthine oxidase enzyme that was pre-incubated with FAD co-factor prior to immobilization into the enzyme matrix. The enzyme used to fabricate the biosensor in Example 38 was pre-incubated with FAD prior to immobilization and said biosensor responded to xanthine analyte. Prior to the application of a generic outer-membrane, the biosensor in Example 38 had a linear response range (>98% linear) of over 50 μM of xanthine analyte.

### EXAMPLES 39, 40 AND 41

Examples 30, 40 and 41 showed biosensors fabricated from oxalate oxidase enzyme. Functional biosensors (as determined by the biosensor response to oxalate analyte) were best achieved when said biosensors were fabricated from oxalate oxidase enzyme that was pre-incubated with Mn²⁺ co-factor prior to immobilization into the enzyme matrix. The enzyme used to fabricate the biosensor in Example 39 was pre-incubated with Mn²⁺ prior to immobilization and said biosensor responded to oxalate analyte. If the enzyme used to fabricate the biosensor in Example 41 was not pre-incubated with Mn²⁺ prior to immobilization and the said biosensor failed to respond to oxalate analyte when tested at the same concentration as Example 39. Prior to the application of a generic outer-membrane, the biosensor in Example 39 had a linear response range (>97% linear) of over 5 mM of oxalate analyte.

Example 39, 40, and 41 also demonstrated that this invention is useful for co-factors that are heavy metals. Entry 40 also demonstrated the importance of using the proper co-factor for complete conditioning and enhancement of enzyme activity. Here, FAD was used instead of Mn²⁺ as the co-factor. Even though a discernible signal was observed, the signal was still five-fold less than that observed for the properly Mn²⁺ conditioned enzyme (Example 39). Example 41 demonstrated that in the absence of any co-factor, the enzyme is once again unable to support catalysis as part of a functioning biosensor.

### EXAMPLE 42

Example 42 showed a biosensor fabricated from cholesterol oxidase enzyme. The functional biosensor (as determined by the biosensor response to cholesterol analyte) was best achieved when said biosensor was fabricated from cholesterol oxidase enzyme that was pre-incubated with FAD co-factor prior to immobilization into the enzyme matrix. The enzyme used to fabricate the biosensor in Example 42 was pre-incubated with FAD prior to immobilization and said biosensor responds to cholesterol analyte.

### EXAMPLES 43, 44, AND 45

Examples 43, 44 and 45 showed biosensors fabricated from gamma-glutamyl-putrascine oxidase enzyme. Functional biosensors (as determined by the biosensor response to GABA analyte) were best achieved when said biosensors were fabricated from gamma-glutamyl-putrascine oxidase enzyme that was pre-incubated with FAD co-factor prior to immobilization into the enzyme matrix. The enzyme used to fabricate the biosensor in Example 43 was pre-incubated with FAD prior to immobilization and said biosensor responded to GABA analyte. The enzyme used to fabricate the biosensor in Example 44 was pre-incubated with FMN prior to immobilization and said biosensor responded to GABA analyte. The enzyme used to fabricate the biosensor in Example 45 was not pre-incubated with co-factor prior to immobilization and said biosensor did not respond to GABA analyte.

### EXAMPLE 46

Example 46 shows a biosensor fabricated from GABA oxidase enzyme. The functional biosensor (as determined by the biosensor response to GABA analyte) is best achieved when said biosensor is fabricated from GABA oxidase enzyme that is pre-incubated with FAD co-factor prior to immobilization into the enzyme matrix. The enzyme that is used to fabricate the biosensor in Example 46 is pre-incubated with FAD prior to immobilization and said biosensor responds to GABA analyte.

### EXAMPLE 47

Example 47 shows a biosensor fabricated from histamine oxidase enzyme (diamine oxidase). The functional biosensor (as determined by the biosensor response to histamine analyte) is best achieved when said biosensor is fabricated from histamine oxidase enzyme that is pre-incubated with FAD co-factor prior to immobilization into the enzyme matrix. The enzyme that is used to fabricate the biosensor in Example 47 is pre-incubated with FAD prior to immobilization and said biosensor responds to histamine analyte.

### EXAMPLE 48

Example 48 shows a biosensor fabricated from nucleoside oxidase enzyme. The functional biosensor (as determined by the biosensor response to adenosine analyte) is best achieved when said biosensor is fabricated from nucleoside oxidase enzyme that is pre-incubated with FAD co-factor prior to immobilization into the enzyme matrix. The enzyme that is used to fabricate the biosensor in Example 48 is pre-incubated with FAD prior to immobilization and said biosensor responds to adenosine analyte.

### EXAMPLE 49

Example 49 shows a biosensor fabricated from L-lysine oxidase enzyme. The functional biosensor (as determined by the biosensor response to L-lysine analyte) is best achieved when said biosensor is fabricated from L-lysine oxidase enzyme that is pre-incubated with FAD co-factor prior to immobilization into the enzyme matrix. The enzyme that is used to fabricate the biosensor in Example 49 is pre-incubated with FAD prior to immobilization and said biosensor responds to L-lysine analyte.

### EXAMPLE 50

Example 50 shows a biosensor fabricated from L-aspartate oxidase enzyme. The functional biosensor (as determined by the biosensor response to L-aspartate analyte) is best achieved when said biosensor is fabricated from L-aspartate oxidase enzyme that is pre-incubated with FAD co-factor prior to immobilization into the enzyme matrix. The enzyme that is used to fabricate the biosensor in Example 50 is pre-incubated with FAD prior to immobilization and said biosensor responds to L-aspartate analyte.

### EXAMPLE 51

Example 51 shows a biosensor fabricated from glycine oxidase enzyme. The functional biosensor (as determined by the biosensor response to glycine analyte) is best achieved when said biosensor is fabricated from glycine oxidase enzyme that is pre-incubated with FAD co-factor prior to immobilization into the enzyme matrix. The enzyme that is used to fabricate the biosensor in Example 51 is pre-incubated with FAD prior to immobilization and said biosensor responds to glycine analyte.

### EXAMPLE 52

Example 52 shows a biosensor fabricated from galactose oxidase enzyme. The functional biosensor (as determined by the biosensor response to galactose analyte) is best achieved when said biosensor is fabricated from galactose oxidase enzyme that is pre-incubated with pyrroloquinoline and quinone co-factor prior to immobilization into the enzyme matrix. The enzyme that is used to fabricate the biosensor in Example 52 is pre-incubated with pyrroloquinoline and quinone prior to immobilization and said biosensor responds to galactose analyte.

### EXAMPLES 53-64

The biosensors in Examples 53, 56, 57, 59, 61, and 63, were fabricated on polyphenol film that formed the inner-membrane of the sensing cavity. In each instance, the indicated oxidase enzyme was pre-incubated with the indicated co-factor prior to immobilization into the sensing matrix. These biosensors all responded to analyte as indicated.

The biosensors in Examples 54, 58, 60, 62, and 64 were fabricated on poly-o-cresol film that formed the inner-membrane of the sensing cavity. In each instance, the indicated oxidase enzyme was pre-incubated with the indicated co-factor prior to immobilization into the sensing matrix. These biosensors all responded to analyte as indicated.

For Example 55, the biosensor was fabricated on polypyrrol film that formed the inner-membrane of the sensing cavity. For this biosensor, the lactate oxidase enzyme was pre-incubated with the FMN co-factor prior to immobilization into the sensing matrix. This biosensor responded to lactate analyte as indicated.

It is important to note that this invention is applicable regardless of the range of analyte tested. For example, a biosensor fabricated from lactate oxidase showed improvement when tested with 10 μM, 100 μM, or 1 mM boluses of lactate analyte. D-amino acid oxidase, glutamate oxidase, and L-6-hydroxynicotine oxidase showed improvement when tested with 10 μM boluses of D-serine, glutamate, and nicotine analytes respectively. Alcohol oxidase shows an improvement when tested with 1 mM boluses of ethanol ethanol. The actual range one might use for any particular biosensor will reflect the expected range that needs to be monitored in vivo for the analyte of interest. The other enzyme/analyte combinations in Table 1 further demonstrated that this invention is applicable for a range of analyte concentrations.

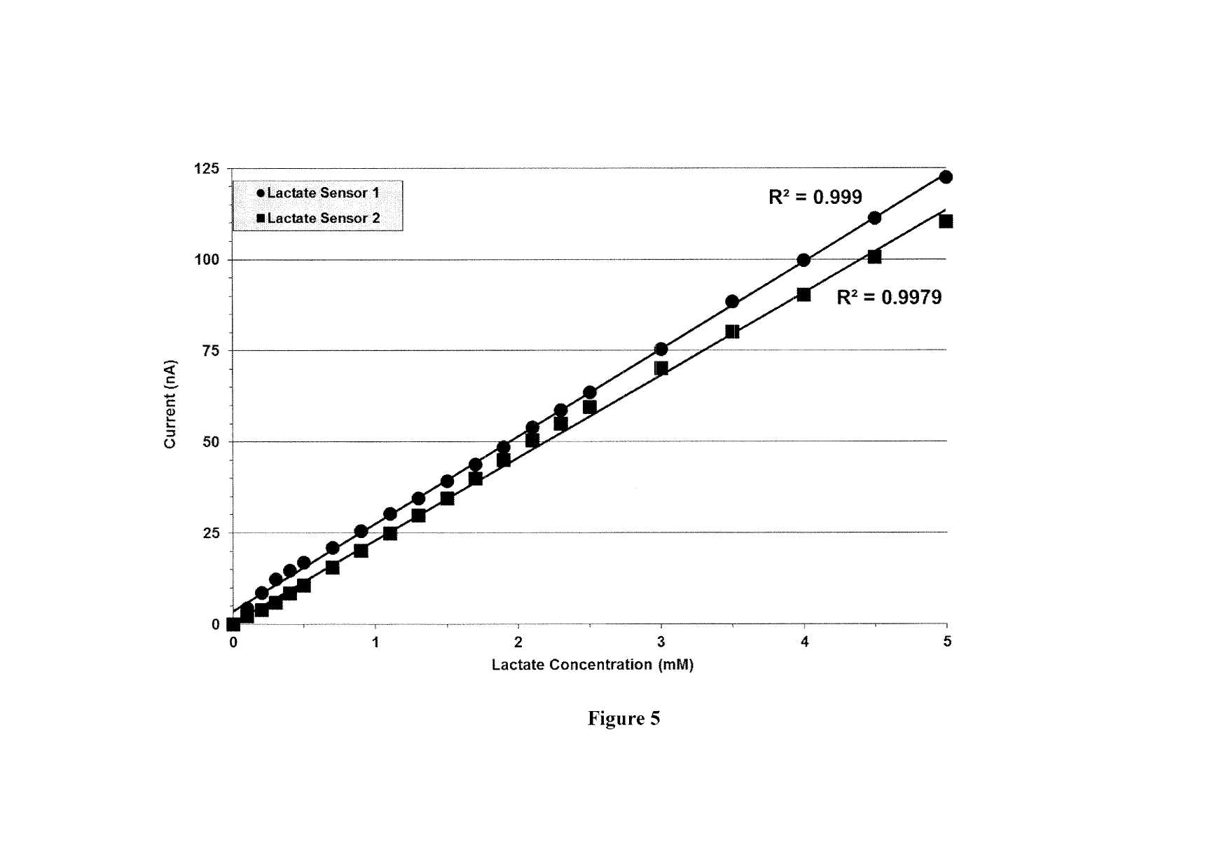

In another embodiment of the invention, biosensors fashioned from enzymes that were conditioned by pre-incubation with the appropriate co-factor(s) showed an increase in shelf stability relative to those biosensors whose biorecognition elements were not conditioned or pre-incubated. This is shown in FIGS. 3 and 4, which speak to the performance of biosensors without pre-incubation with the appropriate co-factors (FIG. 3) versus to those that were pre-incubated with co-factor prior to immobilization (FIG. 4). An improvement in shelf stability was also observed in Entries 16 and 17, where a biosensor was fabricated from properly conditioned D-amino acid oxidase incubated with FAD under standard conditions. After nearly four months, the biosensor retained 73% of its original activity. In another embodiment of the invention, the proper conditioning of an oxidoreductase enzyme by incubation with the appropriate co-factor(s) prior to immobilization also results in biosensors with superior linearity profiles. This was demonstrated with the L-lactate biosensors shown in FIG. 5, which display excellent linearity over a wide concentration range. Most importantly, this concentration range spans the relevant in vivo concentration range that would be needed for an in vivo measurement.

Another embodiment of the invention involves its use for enzymes that can be directed to the measurement of analytes that are not the natural substrate of said enzyme. For example, Entries 18, 20, and 21 demonstrated that L-6-hydroxynicotine oxidase could be used to fabricate a biosensor that would measure nicotine, anabasine, and nor-nicotine even though the natural substrate for the enzyme is 6-hydroxynicotine. This invention is also concerned with the discovery of new co-factor dependent oxidoreductase enzymes that have yet to be described. These new enzymes could be derived from natural sources, by single or multiple mutagenesis changes to an oxidoreductase gene, or by directed evolution using molecular biology techniques of an existing oxidoreductase. These new enzymes may find utility for the sensing of new analytes. According to the invention, these new enzymes may benefit from conditioning with co-factor(s) prior to use as the biorecognition element in a biosensor.

In another embodiment, the invention is useful for co-factor dependent enzymes even if only one species produces such an enzyme, while all other species produce iso-forms that are not co-factor dependent. For example, the human iso-form of diamine oxidase (EC 1.4.3.22), sometimes referred to as histamine oxidase, does not require a co-factor for activity. Instead, human diamine oxidase relies on a post-translational hydroxylation of an active site tyrosine residue to a trihydroxyphenylalanine residue and then finally to a topaquinone (TPQ) residue. By contrast, the diamine oxidase found in rice seedlings from the species Oryza saliva requires FAD as a co-factor for activity (Chaudhuri, M. M.; Ghosh, B. Phytochemistry, 1984, 23, 241-243). Thus, this invention is applicable even if only one species produces an iso-form of a particular oxidoreductase that is co-factor dependent.

In another embodiment of the invention, the utilization of co-factors are not limited to only human iso-forms of a particular enzyme, but also embrace all iso-forms of said enzyme found in all organisms so long as the iso-form is co-factor dependent. It is well accepted and understood that different iso-forms of the same enzyme from different organisms will possess different amino acid sequences and may rely on different co-factors for activity.

In another embodiment, the invention finds utility in activating enzymes where the natural co-factor is normally covalently bonded to the protein, but where the co-factor(s) may have been absent during expression in a natural setting or within a laboratory. This invention may also be used to rescue enzymes whose co-factor(s) was lost during purification of the enzyme, and would be independent of whether the co-factors(s) is non-covalently or covalently bonded.

Enzyme-based biorecognition elements that may benefit from the incubation with co-factor(s) prior to immobilization in a solid matrix include (but are not limited to) malate oxidase, EC 1.1.3.3, co-factor(s)=FAD; hexose oxidase, EC 1.1.3.5, co-factor(s)=Cu; aryl-alcohol oxidase, EC 1.1.3.7, co-factor(s)=FAD; L-gulonolactone oxidase, EC 1.1.3.8, co-factor(s)=FAD; pyranose oxidase, EC 1.1.3.10, co-factor(s)=FAD; L-sorbose oxidase, EC 1.1.3.11, co-factor(s)=FAD(?)(?); pyridoxine 4-oxidase, EC 1.1.3.12, co-factor(s)=FAD; (S)-2-hydroxy-acid oxidase, EC 1.1.3.15, co-factor(s)=FAD, FMN; ecdysone oxidase, EC 1.1.3.16, co-factor(s)=NADPH; secondary-alcohol oxidase, EC 1.1.3.18, co-factor(s)=FAD(?)(?); 4-hydroxymandelate oxidase, EC 1.1.3.19, co-factor(s)=co-factor(s)=FAD(?)(?), Mn2+; long-chain-alcohol oxidase, EC 1.1.3.20, co-factor(s)=FAD; thiamine oxidase, EC 1.1.3.23, co-factor(s)=FAD; hydroxyphytanate oxidase, EC 1.1.3.27. co-factor(s)=FAD(?)(?); N-acylhexosamine oxidase, EC 1.1.3.29, co-factor(s)=FAD(?)(?); polyvinyl-alcohol oxidase, EC 1.1.3.30, co-factor(s)=FAD(?)(?); D-Arabinono-1,4-lactone oxidase, EC 1.1.3.37, co-factor(s)=FAD; vanillyl-alcohol oxidase, EC 1.1.3.38, co-factor(s)=FAD; D-mannitol oxidase, EC 1.1.3.40, co-factor(s)=FAD(?)(?); alditol oxidase, EC 1.1.3.41, co-factor(s)=FAD; choline dehydrogenase, EC 1.1.99.1, co-factor(s)=Coenzyme Q; gluconate 2-dehydrogenase EC 1.1.99.3, co-factor(s)=FAD; glucooligosaccharide oxidase, EC 1.1.99.133, co-factor(s)=FAD; alcohol dehydrogenase, EC 1.1.99.8, co-factor(s)==PQQ; cellobiose dehydrogenase, EC 1.1.99.18, co-factor(s)=FAD; aldehyde oxidase, EC 1.2.3.1, co-factor(s)=FAD; glyoxylate oxidase, EC 1.2.3.5, co-factor(s)=FAD(?)(?); indole-3-acetaldehyde oxidase, EC 1.2.3.7, co-factor(s)==FAD; aryl-aldehyde oxidase, EC 1.2.3.9, co-factor(s)==ATP, NADPH; retinal oxidase, EC 1.2.3.11, co-factor(s)=FAD; abscisic-aldehyde oxidase, EC 1.2.3.14, co-factor(s)==MoCo; aldehyde ferredoxin oxidoreductase, EC 1.2.7.5, co-factor(s)==Ferredoxin tungsten co-factor; indolepyruvate ferredoxin oxidoreductase, EC 1.2.7.8, co-factor(s)=TPP; aldehyde dehydrogenase, EC 1.2.99.7, co-factor(s)=FE-molybdenum; dihydroorotate oxidase, EC 1.3.3.1, co-factor(s)==FAD-FMN; acyl-CoA oxidase, EC 1.3.3.6, co-factor(s)=FAD; dihydrouracil oxidase, EC 1.3.3.7, co-factor(s)==FMN; tetrahydroberberine oxidase, EC 1.3.3.8, co-factor(s)==FAD; tryptophan alpha,beta-oxidase, EC 1.3.3.10, co-factor(s)=(?); L-galactonolactone oxidase, EC 1.3.3.12, co-factor(s)=FAD; acyl-CoA dehydrogenase, EC 1.3.99.3, co-factor(s)=FAD; Isoquinoline-1-oxidoreductase, EC 1.3.99.16, co-factor(s)=iron-sulfur centre or molybdopterin cytosine dinucleotide; quinaldate 4-oxidoreductase, EC 1.3.99.18, co-factor(s)=FAD or pterin molybdenum co-factor; D-aspartate oxidase, EC 1.4.3.1, co-factor(s)=FAD; L-amino-acid oxidase, EC 1.4.3.2, co-factor(s)=FAD; monoamine oxidase, EC 1.4.3.4, co-factor(s)==FAD; pyridoxal 5′-phosphate synthase, EC 1.4.3.5, co-factor(s)==FMN; D-glutamate oxidase, EC 1.4.3.7, co-factor(s)=FAD; ethanolamine oxidase, EC 1.4.3.8; putrescine oxidase, EC 1.4.3.10, co-factor(s)=FAD; cyclohexylamine oxidase, EC 1.4.3.12, co-factor(s)=FAD; protein-lysine 6-oxidase, EC 1.4.3.13, co-factor(s)==lysyl-tyrosyl quinone; D-glutamate(D-aspartate) oxidase, EC 1.4.3.15, co-factor(s)=FAD; L-lysine 6-oxidase, EC 1.4.3.20, co-factor(s)=(?); primary-amine oxidase, EC 1.4.3.21, co-factor(s)==2,4,5-trihydroxyphenylalanine quinone; 7-chloro-L-tryptophan oxidase, EC 1.4.3.23, co-factor(s)=FAD; N-methyl-L-amino-acid oxidase, EC 1.5.3.2, co-factor(s)=FAD; non-specific polyamine oxidase, EC 1.5.3.B2, co-factor(s)=FAD; N8-acetylspermidine oxidase (propane-1,3-diamine-forming), EC 1.5.3.B3, co-factor(s)=FAD; N6-methyl-lysine oxidase, EC 1.5.3.4, co-factor(s)=FAD; polyamine oxidase (propane-1,3-diamine-forming), EC 1.5.3.B4, co-factor(s)=FAD; NI-acetylpolyamine oxidase, EC 1.5.3.B5, co-factor(s)=FAD; spermine oxidase, EC 1.5.3.B6, co-factor(s)=FAD; pipecolate oxidase, EC 1.5.3.7, co-factor(s) =FAD; dimethylglycine oxidase, EC 1.5.3.10, co-factor(s)=FAD; polyamine oxidase, EC 1.5.3.11, co-factor(s)=FAD+Fe; Dihydrobenzophenanthridine oxidase, EC 1.5.3.12, co-factor(s)==FAD+Cu; NAD(P)H oxidase, EC 1.6.3.1, co-factor(s)==FAD+HEME+Ca; urate oxidase, EC 1.7.3.3; 3-aci-nitropropanoate oxidase, EC 1.7.3.5, co-factor(s)==FMN; sulfite oxidase, EC 1.83.1, co-factor(s)==HEME, OLYBDOPTERIN; methanethiol oxidase, EC 1.8.3.4; prenylcysteine oxidase, EC 1.8.3.5, co-factor(s)=FAD; L-ascorbate oxidase, EC 1.10.3.3, co-factor(s)==HEME+CU; 3-hydroxyanthranilate oxidase, EC 1.10.3.5, co-factor(s)==Fe; rifamycin-B oxidase, EC 1.10.3.6, co-factor(s)=(?); superoxide dismutase, EC 1.15.1.1, co-factor(s)=(?); reticuline oxidase, EC 1.21.3.3, co-factor(s)=FAD; lactate oxidase, L-EC 1.1.3.15, co-factor(s)=FAD, FMN; D-amino acid oxidase, EC 1.4.3.3, co-factor(s)=FAD; (S)-6-hydroxynicotine oxidase, EC 1.5.3.5, co-factor(s)=FAD; (R)-6-hydroxynicotine oxidase, EC 1.5.3.6, co-factor(s)=FAD; alcohol oxidase, EC 1.1.3.13, co-factor(s)=FAD; pyruvate oxidase, EC 1.2.3.3, co-factor(s)=FAD, TPP; glucose oxidase, EC 1.1.3.4), co-factor(s)=FAD; L-glutamate oxidase, EC 1.4.3.11, co-factor(s)=FAD; acyl coenzyme A oxidase, EC 1.3.3.6, co-factor(s)=FAD; choline Oxidase, EC 1.1.3.17, co-factor(s)=FAD; glutathione sulfhydryl oxidase, EC 1.8.3.3, co-factor(s)=FAD; glycerolphosphate oxidase, EC 1.1.3.21, co-factor(s)=FAD; sarcosine oxidase, EC 1.5.3.1, co-factor(s)=FAD; xanthine oxidase, EC 1.1.3.22, co-factor(s)==FAD; oxalate oxidase, EC 1.2.3.4, co-factor(s)=Mn²⁺; cholesterol oxidase, EC 1.1.3.6, co-factor(s)=FAD; gamma-glutamyl-putrescine oxidase, EC undefined, obtained from Escherichia coli K12, co-factor(s)=FAD—capable of oxidizing GABA; GABA oxidase, EC undefined, obtained from: Penicillium sp. KAIT-M-117, co-factor(s)=FAD(?)(?); histamine oxidase (diamine oxidase), EC 1.4.3.22, co-factor(s)=pyrroloquinoline, quinone, FAD, others; nucleoside oxidase, EC 1.1.3.39, co-factor(s)=FAD; L-lysine oxidase, EC 1.4.3.14, co-factor(s)=FAD; L-aspartate oxidase, EC 1.4.3.16, co-factor(s)=FAD; glycine oxidase, EC 1.4.3.19, co-factor(s)=FAD; galactose oxidase, EC 1.1.3.9, co-factor(s)=pyrroloquinoline, quinone. The (?) entry indicates that the co-factor is either completely unknown, or that the indicated co-factor has not been verified.

A list of preferred members of this group of enzymes include:
1. Lactate oxidase, EC 1.1.3.15 or EC 1.13.12.4, co-factor(s)=FAD: Monitoring of systemic lactate levels has been used as an important marker in trauma care which can be used to determine the best course of patient treatment. Co-measurement with glucose can further complete the metabolic picture for monitoring of diabetic patients. Point-of-care measurement of lactate is an unsolved medical need. The ability to monitor systemic lactate levels would impact patient care. In the brain, lactate levels rise and fall according to energy demands created by neurotransmitter release and reuptake. Increases in systemic lactate during trauma are recognized as important determinants of mortality.
2. D-amino acid oxidase, EC 1.4.3.3, co-factor(s)=FAD: D-serine is the only D-amino acid found in the central nervous systems of mammals. D-serine is synthesized in the brain by serine racemase from L-serine (its enantiomer). D-serine serves as both a neurotransmitter and a gliotransmitter by activating NMDA receptors. D-serine is a potent agonist at the glycine site of the NMDA-type glutamate receptor.
3. (S)-6-hydroxynicotine oxidase, EC 1.5.3.5, co-factor(s)=FAD: (R)-6-hydroxynicotine oxidase, EC 1.5.3.6, co-factor(s)=FAD; Bacteria can catabolize nicotine for utilization as a primary carbon and nitrogen source. L-6-hydroxynicotine oxidase is the second enzyme in this process from the species Arthrobacter nicotinovorans. This enzyme may also find use in the construction of a nicotine biosensor. Nicotine is an alkaloid found in the nightshade family of plants (Solanaceae) that constitutes approximately 0.6-3.0% of the dry weight of tobacco. In low concentrations, nicotine acts as a stimulant in mammals and is the main factor responsible for the dependence-forming properties of tobacco smoking. According to the American Heart Association, nicotine addiction has historically been one of the hardest addictions to break, while the pharmacological and behavioral characteristics that determine tobacco addiction are similar to those determining addiction to heroin and cocaine.
4. Alcohol Oxidase, EC 1.1.3.13, co-factor(s)=FAD; Alcohol addiction is a disease that affects millions of Americans. Elucidation of the neurochemical pathways that lead to the state of addiction as well as understanding how alcohol elicits neurochemical release will lead to better treatment and prevention of alcohol addiction.
5. Pyruvate oxidase, EC 1.2.3.3, co-factor(s)=FAD and thiamine diphosphate: Pyruvic acid (CH₃COCO₂H), also known as pyruvate, is a key intersection in several metabolic pathways. It supplies energy to living cells when oxygen is present (aerobic respiration) and produces lactate when oxygen is lacking (anaerobic respiration).
6. Glucose oxidase, EC 1.1.3.4, co-factor(s)=FAD: Systemic measurement of glucose is vital to the proper monitoring of a diabetic patient. Local intracerebral measurments help unravel the metabolic pathways intertwined with neurotransmitter release and reuptake. Proper real-time measurement of diabetes can be a very effective preventative step that can save billions in health care dollars every year.
7. Glutamate oxidase, EC 1.4.3.11, co-factor(s)=FAD: As the prevailing excitatory neurotransmitter in the brain, glutamate is essential to normalexcitatory synaptic transmission, although excessive amounts of glutamate (due to faulty regulation) can lead to excitotoxic concentrations of glutamate resulting in cell death. Glutamate is also related to disease states such as Parkinson's and Alzheimer's disease.
8. Acyl coenzyme A oxidase, EC 1.3.3.6, co-factor(s)=FAD: Acyl-coA is important to the metabolism of fatty acids.
9. Glutathione sulfhydryl oxidase, EC 1.8.3.3, co-factor(s)=FAD: Glutathione exists in reduced (GSH) and oxidized (GSSG) states. In the reduced state, the thiol group of cysteine is able to donate a reducing equivalent to other unstable molecules, such as reactive oxygen species. In donating an electron, glutathione itself becomes reactive, but readily reacts with another reactive glutathione to form glutathione disulfide (GSSG). The ratio of reduced glutathione to oxidized glutathione within cells is often used as a measure of cellular toxicity. Glutathione has multiple functions including as an endogenous antioxidant, as a neutralizer of free radicals, and as a regulator of the nitric oxide cycle.
10. Choline oxidase, EC 1.1.3.17, co-factor(s)=FAD: Although choline is primarily regarded only as a building block for acetylcholine, it may find utility as a biomarker for increased need or excess of acetylcholine. Choline oxidase can be used in conjunction with acetylcholine esterase (EC 3.1.1.7) for the determination of acetylcholine concentrations. Acetylcholine is the only neurotransmitter of the somatic nervous system and is an important component within the central and peripheral nervous systems. Acetylcholine in the brain is most closely associated with reward arousal and addiction and is linked to REM sleep.
11. Glycerolphosphate oxidase, EC 1.1.3.21, co-factor(s)=FAD: L-glycerol-3-phosphate is a component of glycerophospholipids and is used to rapidly regenerate NAD+ in brain and skeletal muscle cells of mammals.
12. Sarcosine oxidase, EC 1.5.3.1, co-factor(s)=FAD: Sarcosine, also known as N-methylglycine, is an intermediate and byproduct in glycine synthesis and degradation. Sarcosine is found naturally as an intermediate in the metabolism of choline to glycine.
13. Xanthine oxidase, EC 1.1.3.22, co-factor(s)=FAD+Fe: Derivatives of xanthine are used as second tier treatment of asthma (bronchodialator). It is a mild stimulant from which caffeine is derived.
14. Oxalate oxidase, EC 1.2.3.4, co-factor(s)=Mn²⁺: Oxalate (IUPAC: ethanedioate) is the dianion with formula C₂O₄²⁻. Many metal ions form insoluble precipitates with oxalate, a prominent example being calcium oxalate which is the primary constituent of the most common type of kidney stones. Individuals with kidney disorders, gout, rheumatoid arthritis, and certain forms of vulvodynia are typically advised to avoid foods high in oxalic acid. Methods to reduce the oxalate content in food are of current interest.
15. Cholesterol oxidase, EC 1.1.3.6, co-factor(s)=FAD: Cholesterol is an important component in the production of steroid hormones. High levels in blood serum are markers for heart disease.
16. Gamma-glutamyl-putrescine oxidase, EC undefined, obtained from Escherichia coli K12, co-factor(s)=FAD—capable of oxidizing GABA; GABA Oxidase, EC undefined, obtained from: Penicillium sp. KAIT-M-117, co-factor(s)=FAD(?): GABA is considered the most important inhibitory neurotransmitter in the brain. Gaba-ergic receptors typically regulate chloride ion channels. GABA is present in very low concentrations in the brain (low micro-molar range).
17. Histamine oxidase (diamine oxidase), EC 1.4.3.22, co-factor(s)=FAD+Cu (species specific): Histamine is most known for stimulating an inflammatory response but also acts as a neurotransmitter and regulates some physiological functions, such as sleep regulation. Histamine could be a target for systematic or brain monitoring.
18. Nucleoside oxidase, EC 1.1.3.39, co-factor(s)=FAD: Adenosine is a purine nucleoside comprised of a molecule of adenine attached to a ribofuranose moiety via a β-N9-glycosidic bond. Adenosine plays an important role in biochemical processes, such as energy transfer—as adenosine triphosphate (ATP) and adenosine diphosphate (ADP)—as well as in signal transduction as cyclic adenosine monophosphate. It is also an inhibitory neurotransmitter believed to play a role in promoting sleep and suppressing arousal, with levels increasing with each hour an organism is awake.
19. L-Lysine oxidase, EC 1.4.3.14, co-factor(s)=FAD: As an essential amino acid, lysine is not synthesized in animals, hence it must be ingested as lysine or lysine-containing proteins. L-lysine is metabolized in mammals to give acetyl-CoA. L-lysine is a necessary building block for all protein in the body. L-lysine plays a major role in absorbing calcium; building muscle protein; recovering from surgery or sports injuries; and producing the body's hormones, enzymes, and antibodies.
20. L-Aspartate oxidase, EC 1.4.3.16, co-factor(s)=FAD: Aspartate (the conjugate base of aspartic acid) stimulates NMDA receptors, though not as strongly as the amino acid neurotransmitter glutamate does.
21. Glycine oxidase, EC 1.4.3.19, co-factor(s)=FAD: Glycine is an inhibitory neurotransmitter in the central nervous system, especially in the spinal cord, brainstem, and retina. When glycine receptors are activated, chloride enters the neuron via ionotropic receptors, causing an inhibitory postsynaptic potential (IPSP). Glycine is a required co-agonist along with glutamate for NMDA receptors. In contrast to the inhibitory role of glycine in the spinal cord, this behavior is facilitated at the (NMDA) glutaminergic receptors which are excitatory.
22. Galactose oxidase, EC 1.1.3.9, co-factor(s)=Cu²⁺, Cu³⁺, Mn²⁺ and other heavy metal ions: Chronic systemic exposure of mice, rats, and Drosophila to D-galactose causes the acceleration of senescence and has been used as an aging model. Some studies have suggested a possible link between galactose in milk and ovarian cancer. Ongoing studies also suggest that galactose may play a role in treatment of focal segmental glomerulosclerosis—a kidney disease resulting in kidney failure and proteinuria.

The invention also encompasses the discovery of new co-factor dependent oxidoreductases that may find utility in measuring analytes for which no oxidoreductase presently exists. New oxidoreductases with new or altered analyte activities, specificities, and selectivities may be derived from natural sources, derived by the conversion of an existing oxidoreductase via standard molecular biology mutagenesis techniques or derived by the directed evolution of an existing enzyme. The new co-factor dependent oxidoreductase would have activity, selectivity, and specificity for an analyte not presently available. Such analytes that would benefit from the discovery of a new co-factor dependent oxidoreductase are selected from the list consisting of nicotine, caffeine, cocaine, amphetamine, cortisol, corticosterone, dopamine, serotonin, norepinephrine, L-DOPA, GABA, ATP, and acetylcholine. While some of these analytes may be the substrate of an existing oxidoreductase (e.g., (S)-6-hydroxynicotine oxidase has some activity for nicotine; gamma-glutamyl putrisciene oxidase has some activity for GABA), said analytes are not the primary substrate for said oxidoreductase. Furthermore, said activity, selectivity, and specificity do not necessarily mean that the enzyme is suitable for use in biosensor production. The monitoring of these analytes may benefit by the alternation of said enzyme for the purposes of biosensor fabrication.

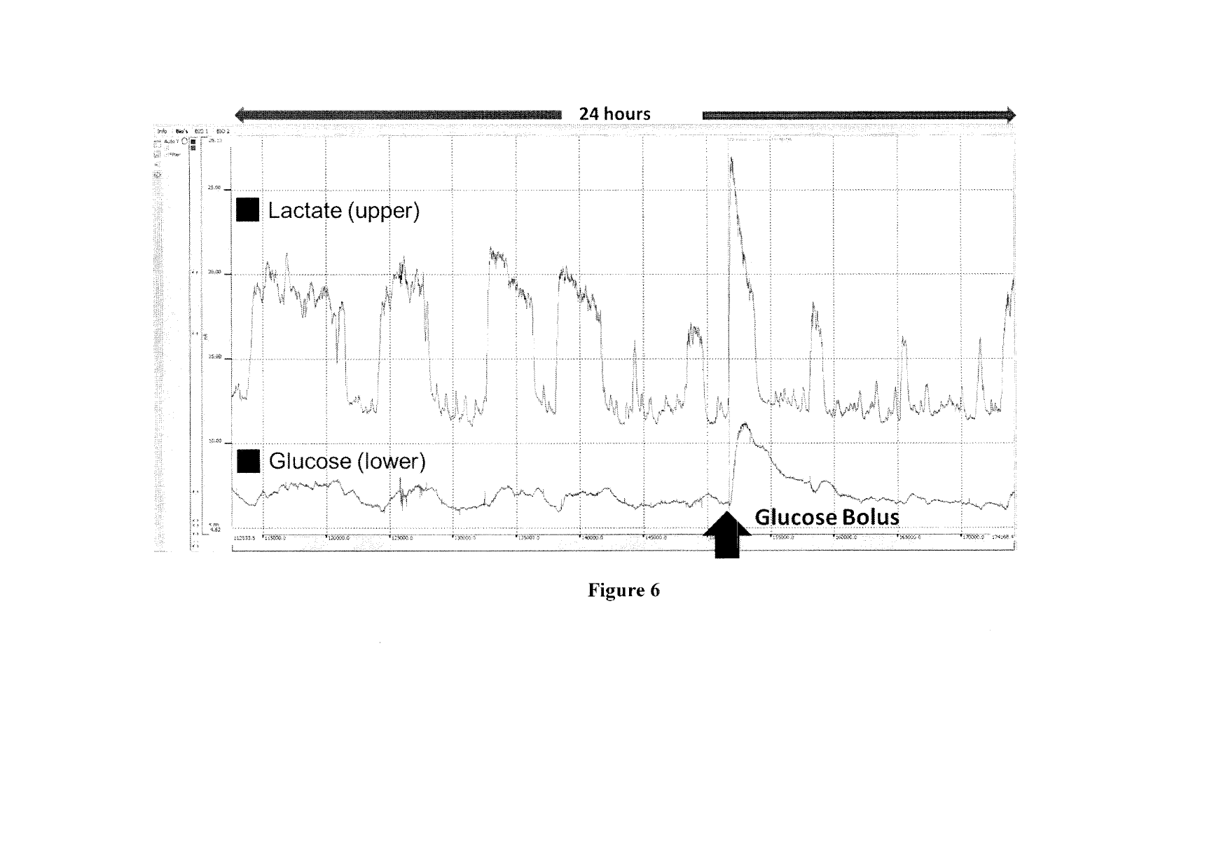

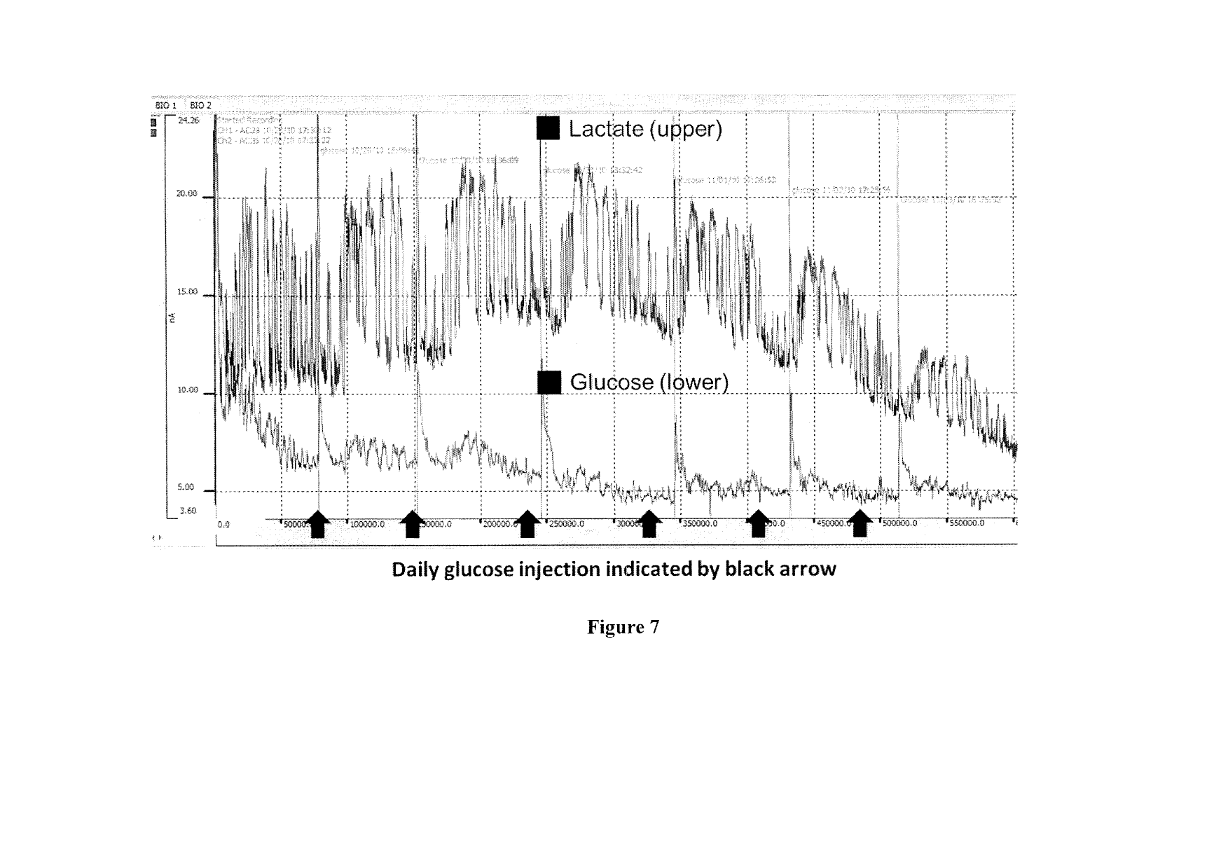

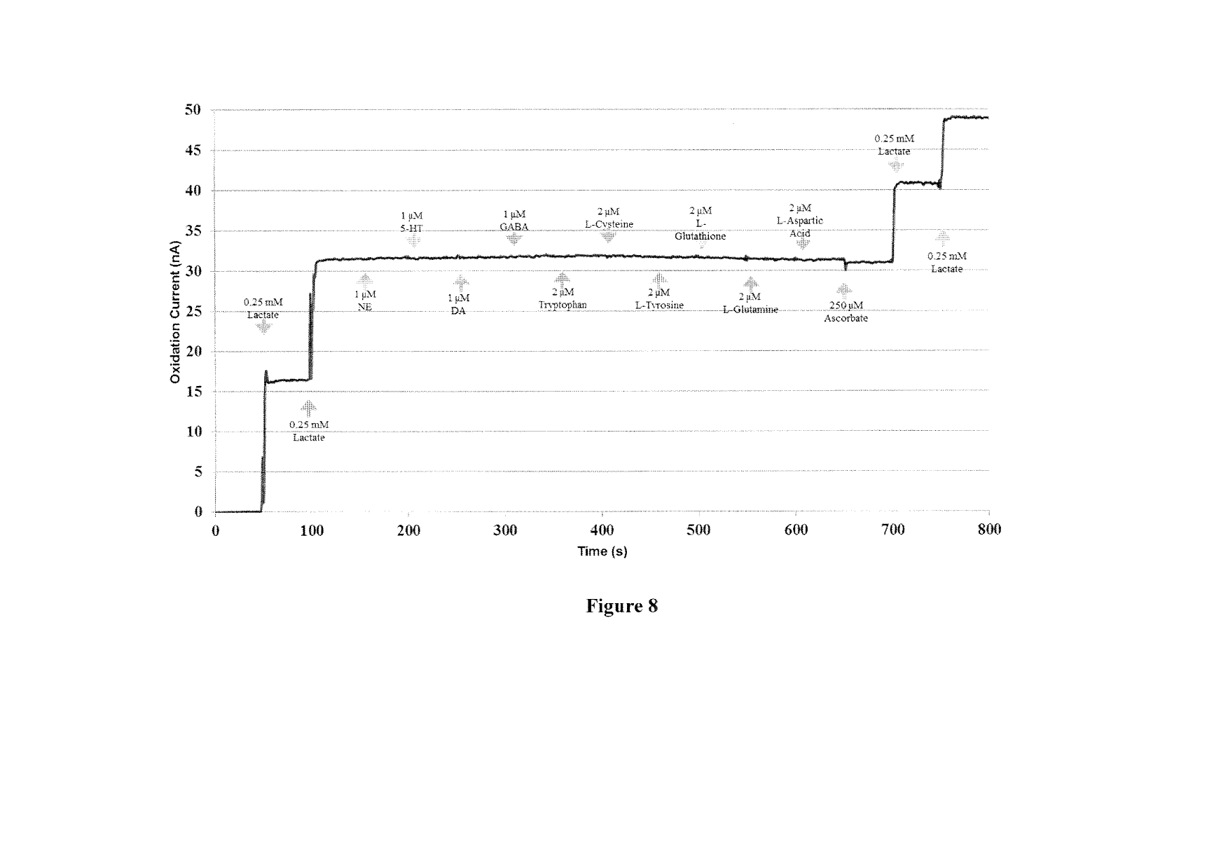

In another embodiment, the invention is completely compatible with all aspects of biosensor fabrication. The fabrication and manufacture of biosensors for commercial applications requires that the biosensor be compatible with the in vivo environment into which it will be placed and must reject endogenous in vivo electroactive interferents that may confound an in vivo measurement. This invention is compatible with existing methods to promote in vivo compatibility, as well as existing methods for excluding potential electroactive interferents. Any process designed to improve the function of the enzyme layer or sensing matrix must be compatible with a biosensor that is fully functional in vivo. This invention, by conditioning an enzyme that will be used as a biosensor's biorecognition element with natural co-factors, is completely compatible with in vivo monitoring of analytes (e.g. glucose and lactate as shown in FIGS. 6 and 7) and preserves the biosensor's ability to reject common electroactive interferents (FIG. 8). This invention is also compatible with methods used to stabilize an enzyme, including mutations. This includes enzymes where the analyte specificity has been altered or evolved and includes co-factor dependent enzymes with dramatically different or altered specificity and selectivity profiles. This invention is also compatible with the addition of adjuncts to the enzyme and enzyme layer. A standard adjunct is bovine serum albumin, which was used in the fabrication of all biosensors described in this invention. This invention is also compatible with co-factor dependent enzymes that are expressed as fusion proteins or co-expressed with chaperones and other molecules that promote proper protein folding.

In another embodiment of the invention, biosensors that are fabricated with pre-incubation of the enzyme with the appropriate co-factor(s) prior to immobilization perform at a stable level in vivo for extended periods of time from days to a week or more. This positive effect on biosensor performance is shown in FIGS. 6 and 7, where a L-lactate and a D-glucose biosensor are contralaterally placed in the brian of a C57B16 mouse. The response to these analytes was observed for one day and seven days. A bolus of glucose was given to the animal every twenty-four hours post-implantation. The response of both biosensors and their respective responses to the daily boluses clearly show that both biosensor remain functional over these time periods. Also note that these biosensors possessed an outer-membrane (Layer 4), demonstrating that this invention is compatible with such a layer.

All enzymes are constantly in motion, and even the most rigid of enzymes have motional and vibrational states that cause atomic level movement within the protein. In general, all oxidoreductases undergo conformation changes as a function of co-factor presence. For oxidoreductases, this process is understood. There exists two limiting conformations, which are reflective of the apo-form of the protein (co-factor(s) absent from the enzyme) and active, holo-form of the protein (co-factor(s) present in the enzyme). The conformations of these two limiting states are distinctly different and can, when immobilized as part of a solid matrix, interact with said matrix differentially.

The act of immobilization establishes the global minimum of the enzyme-matrix system, which is reflective of the predominant state of the enzyme at the time of immobilization. If an enzyme is immobilized as part of a matrix, the form of the enzyme will determine the global thermodynamic minimum of the enzyme-matrix (referred to hereafter as “the system”). If the enzyme is predominantly apo when immobilized as part of the matrix, the thermodynamic minimum for the system reflects the fact that the enzyme is of an apo conformation and an apo shape, and, therefore, the solid matrix surrounding the enzyme interacts and reinforces said conformation. This is reflected in the results from Entries 6, 7, and 8 where the apo enzyme could not be rescued post-immobilization by the addition of co-factor or Entries 10, 11 and 12 where the rescue of the partially holo-enzyme is limited in activity and time.

Other elements of the enzyme layer that may be present further contribute to the stabilization of the apo-enzyme conformation. An energetic price must be paid to force the apo-enzyme into an active, holo-conformation because of the interactions of the enzyme with the surrounding matrix. Thus, while the apo-enzyme can be forced into an active conformation by incubating the system with co-factor(s), the resulting activated form of the enzyme is now at energetic odds with the matrix. Consequently, the system will facilitate the loss of co-factor and revert the enzyme back to the corresponding apo-conformation to re-establish the thermodynamic minimum of the enzyme-matrix system. This is supported with the data from Entries 6, 7 and 8 as well as Entries 14 and 15, Entries 43 and 45and Entries 10, 11, and 12.

Immobilization of enzyme in its active conformation (co-factor(s) present) will result in an active-enzyme/matrix system that reflects the global thermodynamic minimum for the holo-enzyme or active-enzyme conformation. This matrix, built around the active enzyme conformation, promotes efficient enzyme activity. Other elements of the enzyme layer that may be present further contribute to the stabilization of the holo-enzyme conformation. Thus, in comparison to the enzyme-matrix system where the enzyme was predominantly in the apo form, this system is more energetically compatible with the state of the enzyme that is required for catalytic activity and use as the biorecognition element in a biosensor. Note that the enzyme can still lose co-factor even when the thermodynamics of the system are reflective of the active form of the enzyme. It is known that oxidoreductase can readily lose co-factor and that this loss is temperature driven but is reversible. In contrast to the situation in which the system reflects the energetics of the apo conformation, the system that reflects the energetics of the active conformation is more readily reactivated, and the reactivated system is more stable.

An important component of the above, especially with respect to the manufacturability of biosensors is that difficult to handle enzyme preparations can be immobilized en mass for long-term shelf storage and then reactivated prior to use. The importance of the incubation process is independent of the actual biosensor design and is applicable to many designs presently disclosed, including (but not limited to) nano-biosensors, lab-on-chip biosensors, and ceramic biosensors.

Enzymes to be immobilized as biorecognition elements in biosensor construction that are part of this invention are incubated with their co-factor(s) prior to immobilization. Incubation of an enzyme is not limited to a single co-factor, but rather should reflect the optimum interaction of co-factors. Optimization of the enzyme incubation can be determined for each instance of an enzyme and will include the temperature of the incubation, the co-factor(s) concentration, the length of time for incubation, the order of addition of reagents, the ratio of co-factors, and the order of addition and time between additions if multiple co-factors are required for enzyme activity. Even in the absence of optimization of the incubation process, it is expected that any incubation with co-factor(s) will result in an enhancement of the activity of the enzyme which, in turn, will result in an enhancement of the biosensor performance relative to a biosensor whose biorecognition element was not incubated with co-factor(s). The determination of the optimum co-factor enhancement strategy can occur in solution and may be monitored using standard techniques.

This invention allows for the co-immobilization of multiple enzymes simultaneously, which may occur together as part of a single layer or as separate discreet layers that comprise a composite biosensor. In principle, there is no limit to the number of enzymes that function as biorecognition elements that may be co-immobilized. In practical terms, the limit will be determined by the ability of the panoply of enzymes to work in concert with each other. Further, the immobilization of said enzymes could take place on separate regions within a larger array, thereby providing the ability to simultaneously monitor multiple analytes with a single probe. Finally, the mixing of multiple biorecognition elements is not limited to just those that benefit from co-factor enhancement, but rather can reflect a composite group of enzymes that work in concert, at least one of which benefits from said co-factor enhancement. For example, the placement of acetylcholine esterase and choline oxidase as part of the same layer or different layers within a biosensor would allow for the monitoring of acetylcholine levels by the action of (1) conversion of acetylcholine to choline and acetate and (2) the conversion of choline to betaine aldehyde and H₂O₂, the latter of which would be monitored.

This invention is compatible with any number of enzymes, despite their nascent activity prior to immobilization, so long as each enzyme is incubated with the co-factors necessary to insure said enzyme's conversion to the holo-enzyme.

This invention would find utility in a wide range of biosensor designs. Commercially available biosensors that make use of immobilized enzyme(s) as the biorecognition element include the biosensors based on oxidoreductases from Pinnacle Technology Inc. (www.pinnaclet.com) such as those for L-glutamate, D-glucose, L-lactate, and alcohol; sensors based on oxidoreductases from Sarrisa Biomedical (www.sarissa-biomedical.com) such as those for L-lactate, L-glutamate, choline, acetylcholine/choline; sensors based on oxidoreductases from Bluebox Sensors (www.blueboxsensors.com) such as the sensor for D-glucose; and sensors based on oxidoreductases from Quanteon LLC (quanteon.cc) such as those for L-glutamate and choline; sensors based on oxidoreductases from YSI (www.ysilifesciences.com) such as the sensor for D-glucose, sucrose, lactose, L-Lactate, D-galactose, L-glutamate, L-glutamine, choline, ethanol and methanol. Furthermore, the invention of co-factor enhancing the biorecognition element prior to immobilization can work for essentially any biosensor probe design (e.g., planar, cylindrical, disc) or size (about 2 microns to 1000 microns, although in principle there is no lower or upper size limit for which this would apply).

In another embodiment of this invention, the use of co-factor incubation to optimize the performance of the biorecognition element is coupled with an electropolymerization strategy utilizing a phenol precursor to produce a polyphenol film that could serve as either the second layer of a biosensor, the fourth layer of a biosensor, or some combination thereof. In one manifestation of this approach, said film can take the place of polyurethane, providing a more uniform layer that may possess better selectivity properties, better thermal properties, and better mechanical properties. In another manifestation of this approach, the electropolymerization strategy can be utilized to assist in the immobilization of the enzyme. This simultaneously provides the matrix for localization of the biosensing element within the sensing cavity and further provides a film layer that increases the ability of the biosensor to exclude potential interferents. In the extreme case, the polyphenol film layer may preclude the need for other layers as part of the biosensor construction process.

Electropolymerization of phenol precursors to provide a polyphenol film that simultaneously results in the immobilization of the biosensing element can now take complete advantage of the co-factor enhancement process as described. The film will now fully reinforce the shape and consequently the state and conformation of the enzyme by virtue of the nature of the film. For many enzymes that could be utilized as biosensing elements, the stability of the enzyme/co-factor complex in a polyphenol film will far exceed that seen for the same enzyme/co-factor complex in solution. Thus, to maximally exploit the innate nature of a fully activated enzyme, the enzyme should be placed within an environment that helps preserve the active shape, state, and form of the enzyme. Even if loss of co-factor occurs over time, the regeneration of the enzyme's activity, when the enzyme is part of a polyphenol matrix, would be readily accomplished by soaking the biosensor in a solution of co-factor(s) that is consistent with that used in the original biosensor construction.

Polyphenol films may also confer exceptional stability to the biosensor, providing a superior method of crafting amperometric biosensors, most especially those for use in humans. The development of biosensors safe for acute and chronic human applications continues to be an unsolved problem. The utilization of the co-factor enhancement of the biosensing element in conjunction with a polyphenol mediated immobilization of said sensing element now provides for the sensing of a range of analytes that heretofore have been inaccessible.

Examples of biosensors whose biorecognition elements were conditioned by co-factor enhancement prior to immobilization are shown in Entries 46-57 (Table 2). Three different electropolymerized films were tested—polyphenol, poly-o-cresol, and polypyrole—and all three electropolymerized films proved compatible with this invention.

### Implications for a Human Lactate Sensor

The ability to readily determine lactate levels in a new patient presenting with trauma is now recognized as one of the most important diagnostic parameters to help determine the most appropriate course of treatment. Traumas, including brain injuries, shock, sepsis, and haemodynamical instabilities, have all been identified as producing elevated levels of serum lactate. The lactate levels often coincide with the severity of the trauma, and in the case of brain injuries, the magnitude of the insult. Trauma patient morbidity and mortality is known to track with serum lactate levels. Further, decreases in serum lactate levels, as a function of time, can be an early indicator of improved prognoses.

In another embodiment of this invention, a system is developed that provides the ability to determine, in real-time, in vivo lactate levels, including intra-cerebral, sub-cutaneous, intra-muscular, inter-peritoneal, oral, serum, and vascular, the majority of which may act as a surrogate for systemic lactate levels. Such a system would provide a number of distinct improvements to the treatment of soldiers in theater, and the foundation of such a system is a lactate biosensor that has optimal performance characteristics including sensitivity and shelf life. In broad terms, the adaptation of a lactate biosensor of a design described herein for use in humans would provide battlefield medics the ability to appropriately triage soldiers for treatment, most especially those suffering from traumatic brain injuries (TBIs). In the case of TBIs, the speed with which a diagnosis and subsequent treatment options can be made available to medical personnel is often a key mitigant in defining a soldier's long-term prognosis.

The system provides a first responder the ability to monitor, in real-time, the lactate levels of a patient by using a biosensor coupled to recording system (which may be a telemetry system). Telemetry data will be monitorable on essentially any device with wireless capabilities. Long-term accuracy of the sensor, while important, is less critical than delivering acute lactate levels shortly after implantation with a clear indication of how the lactate levels are changing (fast up, fast down). For this application, the biosensor is “pre-charged” to stabilize prior to implantation. Variations (based on biosensor pre-calibration values that are stored in non-volatile memory in the system) are handled via numerical methods embedded within the monitoring software. This system allows for the unprecedented ability to quickly and continuously determine lactate levels as the first step in trauma response and provide a complete history from time of application. A biosensor strategy for monitoring lactate levels offers the following advantages: (1) a highly portable, cost-effective system that would be compatible with complex trauma and battlefield situations, (2) no need for a blood draw with subsequent lab work-up, (3) the elimination of complex blood analysis equipment, and (4) continuous, real-time determination of lactate levels that provide higher temporal resolution than can be provided by traditional draws and work-up. The system also provides for the monitoring of two or more analytes at the same time. For example, in addition to lactate, pyruvate levels are also known to spike in some traumas. Glucose and histamine are also key diagnostic measurements that are known to be useful in defining early treatment strategies and could be measured and monitored in conjunction with lactate levels.

### Experimentals

Lactate oxidase (EC 1.1.3.15 or EC1.13.12.4) was obtained from Genzyme Diagnostics. D-Amino acid oxidase (EC1.4.3.3) was obtained from Calzyme Laboratories or BBI Enzymes. Apo-L-lactate oxidase (EC 1.1,3.15 or EC 1.13.12.4) was obtained as a gift from Professor Mark Richter, University of Kansas. (S)-6-hydroxynicotine oxidase was obtained as a gift from Professor Mark Richter, University of Kansas. Alcohol oxidase (EC 1.1.3.13) was obtained from MP Biomedical. Pyruvate oxidase (EC1.2.3.3) was obtained from Genzyme Diagnostics. L-Glucose oxidase (EC 1.1.3.4) was obtained from BBI Enzymes. L-Glutamate oxidase (EC1.4.3.11) was obtained from Yamasa. Acyl Co-A oxidase (EC 1.3.3.6) was obtained from Genzyme Diagnostics. Choline oxidase (EC 1.1.3.17) was obtained from Asahi Kasei Pharma Corporation. Glutathione Sulthydryl oxidase (EC 1.8.3.3) was obtained from Yamasa. Glycerolphosphate oxidase (EC 1.1.3.21) was obtained from Genzyme Diagnostics. Sarcosine oxidase (EC1.5.3.1) was obtained from Genzyme Diagnostics. Xanthine oxidase (EC 1.17.3.2) was obtained from Diazyme Laboratories. Oxalate oxidase (EC 1.2.3.4) was obtained from Roche Diagnostics. Cholesterol oxidase (EC 1.1.3.6) was obtained from Genzyme Diagnostics. Gamma-Glutaminyl-Putrascine oxidase (EC To be determined) was obtained as a gift from Professor Mark Richter, University of Kansas. Ascorbate oxidase (EC 1.10.3.3) was obtained from Calzyme Laboratories.

Co-factors FAD, FMN, Riboflavin, TPP, and MnCl₂ were all obtained from Sigma-Aldrich. All analytes in Tables 1 and 2 were obtained from commercial sources.

### General Experimental:

For Examples 1-45 (Table 1) and Examples 53-64 (Table 2), the enzyme was dissolved in a 0.1 M solution of the indicated co-factor (in the case of no co-factor, enzymes were dissolved in water) to a nominal concentration of 220 mg/mL.

### Incubation:

Each enzyme solution was allowed to incubate at 0° C. for ninety minutes.

### Immobilization:

Following the incubation period the immobilization mixture was formed by combining (i) 0.35 μL of each enzyme solution, (ii) 0.2 μL of ice cold purified (iii) 0.2 μL of ice cold L-ascorbate oxidase solution, (iv) 0.15 μL of ice cold bovine serum albumin (BSA) solution ([9048-46-8], Sigma) made from 0.4 mg of BSA in 20 μL purified H₂O, and (v) 0.2 μL of ice cold glutaraldehyde solution (0.6 μL glutaraldehyde ([111-30-8], Sigma-Aldrich) in 39.4 μL purified H₂O). The immobilization mixture was then applied to the surface of the sensing cavity. For examples 1-45, a commercially available Pinnacle Technology Inc. #7004-BLANK bare electrode pre-charged with a selective membrane was used. For Examples 53-64, a commercially available Pinnacle Technology Inc. #7004-BLANK bare electrode pre-charged with a selective membrane consisting of the indicated electropolymerized film. When the entire immobilization mixture was consumed, the biosensor was allowed to cure at room temperature. In the case of electrodes made with alcohol oxidase (EC 1.1.3.13), ascorbate oxidase was not included as part of the immobilization mixture.

For Examples 5, 7, 8, 11, 12, and 15, the post-immobilization activation of the enzyme, activation was carried out by incubation of the sensor with the co-factor as described in Table 1 at either 25° C. or 37° C. for a minimum of 30 minutes. This process of post-immobilization activation was usually performed no earlier than 1 day after the enzyme immobilization process as described above.

The biosensors in Examples 1-41, 43-45 and 53-64 were tested in 20 mL of 0.1 M phosphate buffered saline solution. Testing was performed at either ambient or at 37° C. A magnetic stir bar was added to the solution and the container was placed above a magnetic stirrer set to an adequate stirring speed. Biosensors were immersed in the buffer solution and connected to a potentiostat. A bias of 0.6 V with respect to a silver/silver chloride reference electrode (also immersed in the buffered solution) was applied to all electrodes. Once the bias was applied, the sensors were allowed to stabilize (approximately 10 to 20 minutes) and then the analyte for each Example was added to the stirred solution to achieve the indicated concentration within the beaker. All recordings were made on a commercially available Pinnacle Technology Inc. 8400 systems and Pinnacle Technology Inc, 8102 systems.

For Examples 46-52, the enzyme is dissolved in a 0.1 M solution of appropriate co-factor to a nominal concentration of 220 mg/mL.

### Incubation:

Each enzyme solution is allowed to incubate at 0° C. for ninety minutes.

### Immobilization:

Following the incubation period the immobilization mixture is formed by combining (i) 0.35 μL of each enzyme solution, (ii) 0.2 μL of ice cold purified H₂O, (iii) 0.2 μL of ice cold L-ascorbate oxidase solution, (iv) 0.15 μL of ice cold bovine serum albumin (BSA) solution ([9048-46-8], Sigma) made from 0.4 mg of BSA in 20 μL purified H₂O, and (v) 0.2 μL of ice cold glutaraldehyde solution (0.6 μL glutaraldehyde ([111-30-8], Sigma-Aldrich) in 39.4 μL purified H₂O). The immobilization mixture is then applied to the surface of the sensing cavity (e.g, of a commercially available Pinnacle Technology Inc. #7004-BLANK bare electrode pre-charged with a selective membrane or a commercially available Pinnacle Technology Inc. #7004-BLANK bare electrode pre-charged with a selective membrane consisting of an electropolymerized film). When the entire immobilization mixture is consumed, the biosensor is allowed to cure at room temperature. Ascorbate oxidase is included as part of the immobilization mixture as appropriate

The biosensors in Examples 42, 46, 47, 48, 49, 50, 51, and 52 are tested in 20 mL of 0.1 M phosphate buffered saline solution. Testing is performed at either ambient or at 37° C. A magnetic stir bar is added to the solution and the container is placed above a magnetic stirrer set to an adequate stirring speed. Biosensors are immersed in the buffer solution and connected to a potentiostat. A bias of 0.6 V with respect to a silver/silver chloride reference electrode (which is also immersed in the buffered solution) is applied to all electrodes. Once the bias is applied, the sensors are allowed to stabilize (approximately 10 to 20 minutes) and then the analyte for each Example to be tested is added to the stirred solution to achieve the indicated concentration within the beaker. All recordings are made on a commercially available Pinnacle Technology Inc. 8400 systems and Pinnacle Technology Inc. 8102 systems.

For Examples 53, 56, 57, 59, 61 and 63, the polyphenol film was electropolymerized according to the method of Chen (Chen, X. et al.;Biosensors Bioelect., 2002, 17, 1005-1013) onto a commercially available blank sensor (e.g., Pinnacle Technology Inc. #7004-BLANK bare electrode). For Example 55, the polypyrrole film was electropolymerized according to the method of Wassum (Wassum, K. M. et al.; Sensors, 2008, 8, 5023-5036) onto a commercially available blank sensor (e.g., Pinnacle Technology Inc. #7004-BLANK bare electrode). For Examples 54, 58, 60, 62, and 64, the poly-o-cresol film adapted the method of Chen (Chen, X. et al.; Biosensors Bioelect., 2002, 17, 1005-1013) for o-cresol, which was electropolymerized onto a commercially available blank sensor (e.g., Pinnacle Technology Inc. #7004-BLANK bare electrode). All recordings were made on a commercially available Pinnacle Technology Inc. 8400 systems and Pinnacle Technology Inc. 8102 systems.

In vivo bioscnsor recordings were performed on C57B16 male mice obtained from commercial sources (3.9±0.2 months of age, average weight of 29.8±0.9 g). Surgery and biosensors recordings were done according to the published procedure of Naylor et al. (Naylor et al. J. Electroanal, Chem. 2011, 656, 106-113).

TABLE 1

| | | | | Change in | | | |
|---|---|---|---|---|---|---|---|
| | | | | Oxidation Current | | | |
| | | | | (nAmps) to an analyte | | | |
| | Enzyme Identification | | Analyte | bolus in solution | | | |
| Example | (EC Number) | Co-Factor | Tested | 10 uM | 100 uM | 1 mM | Comments |
| 1 | Lactate Oxidase - | None | Lactate | 5.0 | 59.2 | 502.6 | |
| | E.C. 1.1.3.15 or E.C. 1.13.12.7 | | | | | | |
| 2 | Lactate Oxidase - | FMN | Lactate | 12.3 | 146.5 | >1000 | |
| | E.C. 1.1.3.15 or E.C. 1.13.12.4 | | | | | | |
| 3 | Lactate Oxidase - | FAD | Lactate | 9.1 | 113.6 | 915.0 | |
| | E.C. 1.1.3.15 or E.C. 1.13.12.5 | | | | | | |
| 4 | Lactate Oxidase - | Riboflavin | Lactate | 6.1 | 89.2 | 606.0 | |
| | E.C. 1.1.3.15 or E.C. 1.13.12.6 | | | | | | |
| 5 | Lactate Oxidase - | None | Lactate | | | | Soaked in 0.1M FMN for 30 min |
| | E.C. 1.1.3.15 or E.C. 1.13.12.7 | | | | | | post immobilization |
| 6 | Lactate Oxidase - | None | Lactate | | 0.0 | | Pure apo-protein, no co- |
| | E.C. 1.1.3.15 or E.C. 1.13.12.8 | | | | | | factor(s) added pre- |
| | | | | | | | immobilization |
| 7 | Lactate Oxidase - | None | Lactate | | 0.0 | | Soaked in 0.1M FMN for 30 min |
| | E.C. 1.1.3.15 or E.C. 1.13.12.9 | | | | | | post immobilization |
| 8 | Lactate Oxidase - | None | Lactate | | 0.3 | | Soaked in 0.1M FAD for 30 min |
| | E.C. 1.1.3.15 or E.C. 1.13.12.10 | | | | | | post immobilization |
| 9 | Lactate Oxidase - | FMN | Lactate | | 53.0 | | Pure apo-protein, FMN co- |
| | E.C. 1.1.3.15 or E.C. 1.13.12.10 | | | | | | factor added pre- |
| | | | | | | | immobilization |
| 10 | Lactate Oxidase - | None | Lactate | | 8.6 | | Unconditioned protein, no co- |
| | E.C. 1.1.3.15 or E.C. 1.13.12.10 | | | | | | factor pre-immobilization, |
| 11 | Lactate Oxidase - | None | Lactate | | 71.5 | | Entry 10, Soaked in 0.1M |
| | E.C. 1.1.3.15 or E.C. 1.13.12.10 | | | | | | FAD for 30 min post |
| | | | | | | | immobilization, tested after |
| 12 | Lactate Oxidase - | None | Lactate | | 2.0 | | Entry 11, Soaked in 0.1M |
| | E.C. 1.1.3.15 or E.C. 1.13.12.10 | | | | | | FAD for 30 min post |
| | | | | | | | immobilization, after 2 days |
| 13 | D-Amino Acid Oxidase - | FAD | D-Serine | 2.3 | | | |
| | E.C. 1.4.3.3 | | | | | | |
| 14 | D-Amino Acid Oxidase - | None | D-Serine | 0.1 | | | |
| | E.C. 1.4.3.4 | | | | | | |
| 15 | D-Amino Acid Oxidase - | None | D-Serine | 0.7 | | | Soaked in 0.1M FAD for 30 min |
| | E.C. 1.4.3.5 | | | | | | post immobilization |
| 16 | D-Amino Acid Oxidase - | FAD | D-Serine | 1.5 | | | |
| | E.C. 1.4.3.5 | | | | | | |
| 17 | D-Amino Acid Oxidase - | FAD | D-Serine | 1.1 | | | 19+ weeks after initial test |
| | E.C. 1.4.3.5 | | | | | | |
| 18 | 6-hydroxy-L Nicotine Oxidase - | FAD | Nicotine | 0.7 | | | |
| | E.C.1.5.3.5 | | | | | | |
| 19 | 6-hydroxy-L Nicotine Oxidase - | None | Nicotine | 0.0 | | | |
| | E.C.1.5.3.5 | | | | | | |
| 20 | 6-hydroxy-L Nicotine Oxidase - | FAD | Anabasine | 9.8 | | | |
| | E.C.1.5.3.5 | | | | | | |
| 21 | 6-hydroxy-L Nicotine Oxidase - | FAD | Nor-Nicotine | 2.9 | | | |
| | E.C.1.5.3.5 | | | | | | |
| 22 | Alcohol Oxidase - | FAD | Ethanol | | | 31.4 | 1.75 μL of enzyme |
| | EC 1.1.3.13 | | | | | | solution used |
| 23 | Alcohol Oxidase - | None | Ethanol | | | 1.0 | 1.75 μL of enzyme |
| | EC 1.1.3.13 | | | | | | solution used |
| 24 | Pyruvate Oxidase - | FAD & TPP | Pyruvate | | | 0.4 | |
| | E.C.1.2.3.3 | | | | | | |
| 25 | Pyruvate Oxidase - | None | Pyruvate | | | 0.0 | |
| | E.C.1.2.3.3 | | | | | | |
| 26 | Pyruvate Oxidase - | FAD | Pyruvate | | | 0.0 | |
| | E.C.1.2.3.3 | | | | | | |
| 27 | Pyruvate Oxidase - | TPP | Pyruvate | | | 0.0 | |
| | E.C.1.2.3.3 | | | | | | |
| 28 | Pyruvate Oxidase - | FAD | Pyruvate | | | 0.0 | Protein concentration was |
| | E.C.1.2.3.3 | | | | | | 1100 mg/mL |
| 29 | L-Glucose Oxidase - | FAD | Glucose | | | 497.1 | |
| | E.C.1.1.3.4 | | | | | | |
| 30 | L-Glucose Oxidase - | None | Glucose | | | 42.8 | |
| | E.C.1.1.3.4 | | | | | | |
| 31 | L-Glutamate Oxidase - E.C.1.4.3.11 | FAD | Glutamate | 4.7 | | | |
| 32 | L-Glutamate Oxidase - E.C.1.4.3.11 | None | Glutamate | 0.0 | | | |
| 33 | Acyl-CoA Oxidase - | FAD | N-butryl-CoA | 1.6 | | | |
| | E.C.1.3.3.6 | | | | | | |
| 34 | Choline Oxidase - | FAD | Choline | 13.5 | | | |
| | E.C. 1.1.3.17 | | | | | | |
| 35 | Glutathione Sulfhydryl Oxidase - | FAD | Glutathione | 0.1 | 0.6 | 1.9 | |
| | E.C.1.8.3.3 | | | | | | |
| 36 | Glycerolphosphate Oxidase - | FAD | Glycerol-3- | 5.4 | 44.6 | | |
| | E.C. 1.1.3.21 | | Phosphate | | | | |
| 37 | Sarcosine Oxidase - | FAD | Sarcosine | 6.1 | 63.5 | | |
| | E.C.1.5.3.1 | | | | | | |
| 38 | Xanthine Oxidase - | FAD | Xanthine | 1.1 | | | |
| | E.C. 1.17.3.2 | | | | | | |
| 39 | Oxalate Oxidase - | MnCl2 | Oxalic Acid | | | 1.0 | |
| | E.C. 1.2.3.4 | | | | | | |
| 40 | Oxalate Oxidase - | FAD | Oxalic Acid | | | 0.3 | |
| | E.C. 1.2.3.4 | | | | | | |
| 41 | Oxalate Oxidase - | none | Oxalic Acid | | | 0.0 | |
| | E.C. 1.2.3.4 | | | | | | |
| 42 | Cholesterol Oxidase - | FAD | Cholesterol | | | | Not tested due to |
| | E.C. 1.1.3.6 | | | | | | cholesterol insolubility |
| 43 | Gamma-Glutamyl-Putrescine | FAD | GABA | | | 0.5 | |
| | Oxidase - | | | | | | |
| | E.C. To Be Determined | | | | | | |
| 44 | Gamma-Glutamyl-Putrescine | FMN | GABA | | | 0.3 | |
| | Oxidase - | | | | | | |
| | E.C. To Be Determined | | | | | | |
| 45 | Gamma-Glutamyl-Putrescine | None | GABA | | | 0.0 | |
| | Oxidase - | | | | | | |
| | E.C. To Be Determined | | | | | | |
| 46 | GABA Oxidase - | FAD | GABA | | | | |
| | E.C. To Be Determined | | | | | | |
| 47 | Histamine Oxidase - | FAD, | Histamine | | | | |
| | E.C. 1.4.3.22 | pyrroloquinoline, | | | | | |
| | | quinone | | | | | |
| 48 | Nucleoside Oxidase - | FAD | Adenosine | | | | |
| | E.C. 1.1.3.39 | | | | | | |
| 49 | L-Lysine Oxidase - | FAD | L-Lysine | | | | |
| | E.C. 1.4.3.14 | | | | | | |
| 50 | L-Aspartate Oxidase - | FAD | L-Aspartate | | | | |
| | E.C. 1.4.3.16 | | | | | | |
| 51 | Glycine Oxidase - | FAD | Glycine | | | | |
| | E.C. 1.4.3.19 | | | | | | |
| 52 | Galactose Oxidase - | pyrroloquinoline, | D-Galactose | | | | |
| | E.C. 1.1.3.9 | quinone | | | | | |

TABLE 2

| | | | | | Change in | | | |
|---|---|---|---|---|---|---|---|---|
| | | | | | Oxidation Current | | | |
| | | | | | (nAmps) to an analyte | | | |
| | Enzyme Identification | | | Analyte | bolus in solution | | | |
| Example | (EC Number) | Film Type | Co-Factor | Tested | 10 uM | 100 uM | 1 mM | Comments |
| 53 | Lactate Oxidase - | Polyphenol | FAD | Lactate | | 43.0 | | |
| | E.C. 1.1.3.15 or E.C. 1.13.12.7 | | | | | | | |
| 54 | Lactate Oxidase - | Poly-o-cresol | FMN | Lactate | | 22.2 | | |
| | E.C. 1.1.3.15 or E.C. 1.13.12.7 | | | | | | | |
| 55 | Lactate Oxidase - | Polypyrrole | FMN | Lactate | | 283.3 | | |
| | E.C. 1.1.3.15 or E.C. 1.13.12.7 | | | | | | | |
| 56 | Alcohol Oxidase - | Polyphenol | FAD | Ethanol | | | 56.2 | 0.4 μL of a 24 mg/mL |
| | EC 1.1.3.13 | | | | | | | enzyme solution |
| 57 | L-Glucose Oxidase - | Polyphenol | FAD | Glucose | | | 209.7 | |
| | E.C.1.1.3.4 | | | | | | | |
| 58 | L-Glucose Oxidase - | Poly-o-cresol | FAD | Glucose | | | 91.4 | |
| | E.C.1.1.3.4 | | | | | | | |
| 59 | L-Glutamate Oxidase - | Polyphenol | FAD | Glutamate | 1.3 | | | |
| | E.C.1.4.3.11 | | | | | | | |
| 60 | L-Glutamate Oxidase - | Poly-o-cresol | FAD | Glutamate | 0.1 | | | |
| | E.C.1.4.3.11 | | | | | | | |
| 61 | Choline Oxidase - | Polyphenol | FAD | Choline | 0.4 | | | |
| | E.C. 1.1.3.17 | | | | | | | |
| 62 | Choline Oxidase - | Poly-o-cresol | FAD | Choline | 0.6 | | | |
| | E.C. 1.1.3.17 | | | | | | | |
| 63 | Sarcosine Oxidase - | Polyphenol | FAD | Sarcosine | 1.3 | 15.5 | 90.6 | |
| | E.C.1.5.3.1 | | | | | | | |
| 64 | Sarcosine Oxidase - | Poly-o-cresol | FAD | Sarcosine | 0.8 | 5.7 | 29.0 | |
| | E.C.1.5.3.1 | | | | | | | |

We claim:

1. A method of forming a sensor, said method comprising:
incubating an enzyme in the presence of a co-factor for said enzyme; and
immobilizing said enzyme in its active form.

2. The method of claim 1, wherein said incubating is carried out at a temperature of from about −10° C. to about 100° C.

3. The method of claim 1, wherein said incubating is carried out for a time period of from about 1 minute to about 72 hours.

4. The method of claim 1, wherein said enzyme is an enzyme that produces electroactive byproducts.

5. The method of claim 4, wherein said enzyme is selected from the group consisting of flavoproteins.

6. The method of claim 5, wherein said enzyme is selected from the group consisting of oxidoreductases and reductases.

7. The method of claim 4, wherein said enzyme is selected from the group consisting of malate oxidase, hexose oxidase, aryl-alcohol oxidase, L-gulonolactone oxidase, pyranose oxidase, L-sorbose oxidase, pyridoxine 4-oxidase (S)-2-hydroxy-acid oxidase, ecdysone oxidase, secondary-alcohol oxidase, 4-hydroxymandelate oxidase, long-chain-alcohol oxidase, thiamine oxidase, hydroxyphytanate oxidase, N-acylhexosamine oxidase, polyvinyl-alcohol oxidase, D-arabinono-1,4-lactone oxidase, vanillyl-alcohol oxidase, D-mannitol oxidase, alditol oxidase, choline dehydrogenase, gluconate 2-dehydrogenase, glucooligosaccharide oxidase, alcohol dehydrogenase, cellobiose dehydrogenase, aldehyde oxidase, glyoxylate oxidase, indole-3-acetaldehyde oxidase, aryl-aldehyde oxidase, retinal oxidase, abscisic-aldehyde oxidase, aldehyde ferredoxin oxidoreductase, indolepyruvate ferredoxin oxidoreductase, aldehyde dehydrogenase, dihydroorotate oxidase, dihydrouracil oxidase, tetrahydroberberine oxidase, tryptophan alpha,beta-oxidase, L-galactonolactone oxidase, acyl-CoA dehydrogenase, isoquinoline 1-oxidoreductase, quinaldate 4-oxidoreductase, D-aspartate oxidase, L-amino-acid oxidase, monoamine oxidase, pyridoxal 5′-phosphate synthase, D-glutamate oxidase, ethanolamine oxidase, putrescine oxidase, cyclohexylamine oxidase, protein-lysine 6-oxidase, D-glutamate(D-aspartate) oxidase, L-lysine 6-oxidase, primary-amine oxidase, 7-chloro-L-tryptophan oxidase, N-methyl-L-amino-acid oxidase, non-specific polyamine oxidase, N8-acetylspermidine oxidase (propane-1,3-diamine-forming), N6-methyl-lysine oxidase, polyamine oxidase (propane-1,3-diamine-forming), N1-acetylpolyamine oxidase, spermine oxidase, L-pipecolate oxidase, dimethylglycine oxidase, polyamine oxidase, E-dihydrobenzophenanthridine oxidase, NAD(P)H oxidase, urate oxidase, aci-nitropropanoate oxidase, sulfite oxidase, methanethiol oxidase, prenylcysteine oxidase, L-ascorbate oxidase, 3-hydroxyanthranilate oxidase, rifamycin-B oxidase, superoxide dismutase, reticuline oxidase, lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

8. The method of claim 7, wherein said enzyme is selected from the group consisting of lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

9. The method of claim 1, wherein said incubating is carried out in the presence of a plurality of co-factors for said enzyme.

10. The method of claim 1, wherein said immobilizing comprises immobilizing said enzyme in a matrix in its active form.

11. The method of claim 10, wherein said immobilizing comprises crosslinking said enzyme with said matrix.

12. The method of claim 10, wherein said immobilizing comprises entrapping said enzyme in said matrix.

13. The method of claim 10, wherein said immobilizing comprises electropolymerizing a matrix precursor with said enzyme.

14. The method of claim 10, wherein said matrix is selected from the group consisting of low molecular weight bis-aldehydes, glutaraldehyde and serum albumen, polyphenols, polypyrroles, polyphenylenediamines, polyurethanes, sulfonated polytetrafluoroethylenes, cellulose acetates, sol gels, and zeolites.

15. The method of claim 1, wherein said co-factor is selected from the group consisting of FAD, FMN, thiamine pyrophosphate, quinone, heme, porphorin, and heavy metals.

16. The method of claim 1, wherein said immobilization comprises adhering said enzyme in its active form to a support.

17. The method of claim 1, wherein said immobilizing comprises immobilizing said enzyme in a matrix in its active form to a support.

18. The method of claim 17, wherein said support comprises a body selected from the group consisting of cylindrical bodies and planar bodies.

19. A sensing element comprising:
a support having a surface; and
a layer on said surface, said layer comprising an enzyme and a co-factor for said enzyme in a matrix, said enzyme being predominantly in its active form and said matrix retaining said enzyme in said active form.

20. The sensing element of claim 19, said enzyme being an enzyme other than glucose oxidase.

21. The sensing element of claim 19, said enzyme being an enzyme other than glutamate oxidase.

22. The sensing element of claim 19, wherein said enzyme is an enzyme that produces electroactive byproducts.

23. The sensing element of claim 22, wherein said enzyme is selected from the group consisting of flavoproteins.

24. The sensing element of claim 23. wherein said enzyme is selected from the group consisting of oxidoreductases and reductases.

25. The sensing element of claim 22, wherein said enzyme is selected from the group consisting of malate oxidase, hexose oxidase, aryl-alcohol oxidase, L-gulonolactone oxidase, pyranose oxidase, L-sorbose oxidase, pyridoxine 4-oxidase (S)-2-hydroxy-acid oxidase, ecdysone oxidase, secondary-alcohol oxidase, 4-hydroxymandelate oxidase, long-chain-alcohol oxidase, thiamine oxidase, hydroxyphytanate oxidase, N-acylhexosamine oxidase, polyvinyl-alcohol oxidase, D-arabinono-1,4-lactone oxidase, vanillyl-alcohol oxidase, D-mannitol oxidase, alditol oxidase, choline dehydrogenase, gluconate 2-dehydrogenase, glucooligosaccharide oxidase, alcohol dehydrogenase, cellobiose dehydrogenase, aldehyde oxidase, glyoxylate oxidase, indole-3-acetaldehyde oxidase, aryl-aldehyde oxidase, retinal oxidase, abscisic-aldehyde oxidase, aldehyde ferredoxin oxidoreductase, indolepyruvate ferredoxin oxidoreductase, aldehyde dehydrogenase, dihydroorotate oxidase, dihydrouracil oxidase, tetrahydroberberine oxidase, tryptophan alpha,beta-oxidase, L-galactonolactone oxidase, acyl-CoA dehydrogenase, isoquinoline I -oxidoreductase, quinaldate 4-oxidoreductase, D-aspartate oxidase, L-amino-acid oxidase, monoamine oxidase, pyridoxal 5′-phosphate synthase, D-glutamate oxidase, ethanolamine oxidase, putrescine oxidase, cyclohexylamine oxidase, protein-lysine 6-oxidase, D-glutamate(D-aspartate) oxidase, L-lysine 6-oxidase, primary-amine oxidase, 7-chloro-L-tryptophan oxidase, N-methyl-L-amino-acid oxidase, non-specific polyamine oxidase, N8-acetylspermidine oxidase (propane-1,3-diamine-forming), N6-methyl-lysine oxidase, polyamine oxidase (propane-1,3-diamine-forming), N1-acetylpolyamine oxidase, spermine oxidase, L-pipecolate oxidase, dimethylglycine oxidase, polyamine oxidase, E-dihydrobenzophenanthridine oxidase, NAD(P)H oxidase, urate oxidase, aci-nitropropanoate oxidase, sulfite oxidase, methanethiol oxidase, prenylcysteine oxidase, L-ascorbate oxidase, 3-hydroxyanthranilate oxidase, rifamycin-B oxidase, superoxide dismutase, reticuline oxidase, lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

26. The sensing element of claim 25, wherein said enzyme is selected from the group consisting of lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

27. The sensing element of claim 19, wherein said layer comprises a plurality of co-factors for said enzyme.

28. The sensing element of claim 19, wherein said enzyme is crosslinked with said matrix.

29. The sensing element of claim 19, wherein said enzyme is entrapped in said matrix.

30. The sensing element of claim 19, wherein said enzyme is polymerized with said matrix.

31. The sensing element of claim 19, wherein said matrix is selected from the group consisting of low molecular weight bis-aldehydes, glutaraldehyde and serum albumen, polyphenols, polypyrroles, polyphenylenediamines, polyurethanes, sulfonated polytetrafluoroethylenes, cellulose acetates, sol gels, and zeolites.

32. The sensing element of claim 19, wherein said co-factor is selected from the group consisting of FAD, FMN, thiamine pyrophosphate, quinone, home, porphorin, and heavy metals.

33. The sensing element of claim 19, wherein said support comprises a body selected from the group consisting of cylindrical bodies and planar bodies.

34. The sensing element of claim 19, said matrix retaining said enzyme in said active form for at least about 24 hours.

35. The sensing element of claim 34, said matrix retaining said enzyme in said active form for at least about 48 hours.

36. The sensing element of claim 35, said matrix retaining said enzyme in said active form for at least about 72 hours.

37. The sensing element of claim 36, said matrix retaining said enzyme in said active form for at least about 1 week.

38. The sensing element of claim 37, said matrix retaining said enzyme in said active form for at least about 2 weeks.

39. The sensing element of claim 38, said matrix retaining said enzyme in said active form for at least about 3 weeks.

40. The sensing element of claim 39, said matrix retaining said enzyme in said active form for at least about 4 weeks.

41. The sensing element of claim 40, said matrix retaining said enzyme in said active form for at least about 8 weeks.

42. The sensing element of claim 41, said matrix retaining said enzyme in said active form for at least about 12 weeks.

43. The sensing element of claim 42, said matrix retaining said enzyme in said active form for at least about 16 weeks.

44. The sensing element of claim 43, said matrix retaining said enzyme in said active form for at least about 30 weeks.

45. The sensing element of claim 44, said matrix retaining said enzyme in said active form for at least about 52 weeks.

46. An amperometric biosensor comprising:
a working electrode comprising a sensing element comprising:
a support having a surface; and
a layer on said surface, said layer comprising an enzyme and a co-factor for said enzyme in a matrix, said enzyme being predominantly in its active form and said matrix retaining said enzyme in said active form; and
a reference electrode.

47. The biosensor of claim 46, further comprising a counter electrode.

48. The biosensor of claim 46, wherein said working electrode has a diameter of from about 2 μm to about 500 μm.

49. The biosensor of claim 46, wherein said working electrode is formed from a material selected from the group consisting of platinum-iridium, platinum, carbon, stainless steel, nitinol, tungsten, cadmium, and gold.

50. The biosensor of claim 46, wherein said reference electrode is formed from Ag/AgCl.

51. The biosensor of claim 46, said enzyme being an enzyme other than glucose oxidase.

52. The biosensor of claim 46, said enzyme being an enzyme other than glutamate oxidase.

53. The biosensor of claim 46, wherein said enzyme is an enzyme that produces electroactive byproducts.

54. The biosensor of claim 46, wherein said enzyme is selected from the group consisting of flavoproteins.

55. The biosensor of claim 54, wherein said enzyme is selected from the group consisting of oxidoreductases and reductases.

56. The biosensor of claim 53, wherein said enzyme is selected from the group consisting of malate oxidase, hexose oxidase, aryl-alcohol oxidase, L-gulonolactone oxidase, pyranose oxidase, L-sorbose oxidase, pyridoxine 4-oxidase (S)-2-hydroxy-acid oxidase, ecdysone oxidase, secondary-alcohol oxidase, 4-hydroxymandelate oxidase, long-chain-alcohol oxidase, thiamine oxidase, hydroxyphytanate oxidase, N-acylhexosamine oxidase, polyvinyl-alcohol oxidase, D-arabinono-1,4-lactone oxidase, vanillyl-alcohol oxidase, D-mannitol oxidase, alditol oxidase, choline dehydrogenase, gluconate 2-dehydrogenase, glucooligosaccharide oxidase, alcohol dehydrogenase, cellobiose dehydrogenase, aldehyde oxidase, glyoxylate oxidase, indole-3-acetaldehyde oxidase, aryl-aldehyde oxidase, retinal oxidase, abscisic-aldehyde oxidase, aldehyde ferredoxin oxidoreductase, indolepyruvate ferredoxin oxidoreductase, aldehyde dehydrogenase, dihydroorotate oxidase, dihydrouracil oxidase, tetrahydroberberine oxidase, tryptophan alpha,beta-oxidase, L-galactonolactone oxidase, acyl-CoA dehydrogenase, isoquinoline 1-oxidoreductase, quinaldate 4-oxidoreductase, D-aspartate oxidase, L-amino-acid oxidase, monoamine oxidase, pyridoxal 5′-phosphate synthase, D-glutamate oxidase, ethanolamine oxidase, putrescine oxidase, cyclohexylamine oxidase, protein-lysine 6-oxidase, D-glutamate(D-aspartate) oxidase, L-lysine 6-oxidase, primary-amine oxidase, 7-chloro-L-tryptophan oxidase, N-methyl-L-amino-acid oxidase, non-specific polyamine oxidase, N8-acetylspermidine oxidase (propane-1,3-diamine-forming), N6-methyl-lysine oxidase, polyamine oxidase (propane-1,3-diamine-forming), N1-acetylpolyamine oxidase, spermine oxidase, L-pipecolate oxidase, dimethylglycine oxidase, polyamine oxidase, E-dihydrobenzophenanthridine oxidase, NAD(P)H oxidase, urate oxidase, aci-nitropropanoate oxidase, sulfite oxidase, methanethiol oxidase, prenylcysteine oxidase, L-ascorbate oxidase, 3-hydroxyanthranilate oxidase, rifamycin-B oxidase, superoxide dismutase, reticuline oxidase, lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

57. The biosensor of claim 56, wherein said enzyme is selected from the group consisting of lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

58. The biosensor of claim 46, wherein said layer comprises a plurality of co-factors for said enzyme.

59. The biosensor of claim 46, wherein said enzyme is crosslinked with said matrix.

60. The biosensor of claim 46, wherein said enzyme is entrapped in said matrix.

61. The biosensor of claim 46, wherein said enzyme is polymerized with said matrix.

62. The biosensor of claim 46, wherein said matrix is selected from the group consisting of low molecular weight bis-aldehydes, glutaraldehyde and serum albumen, polyphenols, polypyrroles, polyphenylenediamines, polyurethanes, sulfonated polytetrafluoroethylenes, cellulose acetates, sol gels, and zeolites.

63. The biosensor of claim 46, wherein said co-factor is selected from the group consisting of FAD, FMN, thiamine pyrophosphate, quinone, heme, porphorin, and heavy metals.

64. The biosensor of claim 46, wherein said support comprises a body selected from the group consisting of cylindrical bodies and planar bodies.

65. The biosensor of claim 46, said matrix retaining said enzyme in said active form for at least about 24 hours.

66. The biosensor of claim 65, said matrix retaining said enzyme in said active form for at least about 48 hours.

67. The biosensor of claim 66, said matrix retaining said enzyme in said active form for at least about 72 hours.

68. The biosensor of claim 67, said matrix retaining said enzyme in said active form for at least about 1 week.

69. The biosensor of claim 68, said matrix retaining said enzyme in said active form for at least about 2 weeks.

70. The biosensor of claim 69, said matrix retaining said enzyme in said active form for at least about 3 weeks.

71. The biosensor of claim 70, said matrix retaining said enzyme in said active form for at least about 4 weeks.

72. The biosensor of claim 71, said matrix retaining said enzyme in said active form for at least about 8 weeks.

73. The biosensor of claim 72, said matrix retaining said enzyme in said active form for at least about 12 weeks.

74. The biosensor of claim 73, said matrix retaining said enzyme in said active form for at least about 16 weeks.

75. The biosensor of claim 74, said matrix retaining said enzyme in said active form for at least about 30 weeks.

76. The biosensor of claim 75, said matrix retaining said enzyme in said active form for at least about 52 weeks.

77. An assembly for oral, intra-cerebral, intramuscular, intravascular, vascular, inter-peritoneal, or sub-cutaneous placement and anchoring, said assembly comprising:
an amperometric biosensor comprising:
a working electrode comprising a sensing element comprising:
a support having a surface; and
a layer on said surface, said layer comprising an enzyme and a co-factor for said enzyme in a matrix, said enzyme being predominantly in its active form and said matrix retaining said enzyme in said active form; and
a reference electrode; and
a device selected from the group consisting of a cannula, a cannula headpiece, a patch, an implant, a trocar, said biosensor being attached to said device.

78. The assembly of claim 77, said biosensor further comprising a counter electrode.

79. The assembly of claim 77, wherein said working electrode has a diameter of from about 2 μm to about 500 μm.

80. The assembly of claim 77, wherein said working electrode is formed from a material selected from the group consisting of platinum-iridium, platinum, carbon, stainless steel, nitinol, tungsten, cadmium, and gold.

81. The assembly of claim 77, wherein said reference electrode is formed from Ag/AgCl.

82. The assembly of claim 77, said enzyme being an enzyme other than glucose oxidase.

83. The assembly of claim 77, said enzyme being an enzyme other than glutamate oxidase.

84. The assembly of claim 77, wherein said enzyme is an enzyme that produces electroactive byproducts.

85. The assembly of claim 84, wherein said enzyme is selected from the group consisting of flavoproteins.

86. The assembly of claim 85, wherein said enzyme is selected from the group consisting of oxidoreductases and red uctases.

87. The assembly of claim 84, wherein said enzyme is selected from the group consisting of malate oxidase, hexose oxidase, aryl-alcohol oxidase, L-gulonolactone oxidase, pyranose oxidase, L-sorbose oxidase, pyridoxine 4-oxidase (S)-2-hydroxy-acid oxidase, ecdysone oxidase, secondary-alcohol oxidase, 4-hydroxymandelate oxidase, long-chain-alcohol oxidase, thiamine oxidase, hydroxyphytanate oxidase, N-acylhexosamine oxidase, polyvinyl-alcohol oxidase, D-arabinono-1,4-lactone oxidase, vanillyl-alcohol oxidase, D-mannitol oxidase, alditol oxidase, choline dehydrogenase, gluconate 2-dehydrogenase, glucooligosaccharide oxidase, alcohol dehydrogenase, cellobiose dehydrogenase, aldehyde oxidase, glyoxylate oxidase, indole-3-acetaldehyde oxidase, aryl-aldehyde oxidase, retinal oxidase, abscisic-aldehyde oxidase, aldehyde ferredoxin oxidoreductase, indolepyruvate ferredoxin oxidoreductase, aldehyde dehydrogenase, dihydroorotate oxidase, dihydrouracil oxidase, tetrahydroberberine oxidase, tryptophan alpha,beta-oxidase, L-galactonolactone oxidase, acyl-CoA dehydrogenase, isoquinoline 1-oxidoreductase, quinaldate 4-oxidoreductase, D-aspartate oxidase, amino-acid oxidase, monoamine oxidase, pyridoxal 5′-phosphate synthase, D-glutamate oxidase, ethanolamine oxidase, putrescine oxidase, cyclohexylamine oxidase, protein-lysine 6-oxidase, D-glutamate(D-aspartate) oxidase, L-lysine 6-oxidase, primary-amine oxidase, 7-chloro-L-tryptophan oxidase, N-methyl-L-amino-acid oxidase, non-specific polyamine oxidase, N8-acetylspermidine oxidase (propane-1,3-diamine-forming), N6-methyl-lysine oxidase, polyamine oxidase (propane-1,3-diamine-forming), N1-acetylpolyam ine oxidase, spermine oxidase, L-pipecolate oxidase, dimethylglycine oxidase, polyamine oxidase, E-dihydrobenzophenanthridine oxidase, NAD(P)H oxidase, urate oxidase, aci-nitropropanoate oxidase, sulfite oxidase, methanethiol oxidase, prenylcysteine oxidase, L-ascorbate oxidase, 3-hydroxyanthran date oxidase, rifamycin-B oxidase, superoxide dismutase, reticuline oxidase, lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

88. The assembly of claim 87, wherein said enzyme is selected from the group consisting of lactate oxidase, D-amino acid oxidase, (S)-6-hydroxynicotine oxidase, (R)-6-hydroxynicotine oxidase, alcohol oxidase, pyruvate oxidase, glucose oxidase, glutamate oxidase, acyl coenzyme A oxidase, choline oxidase, glutathione sulfhydryl oxidase, glycerolphosphate oxidase, sarcosine oxidase, xanthine oxidase, oxalate oxidase, cholesterol oxidase, gamma-glutamyl-putrescine oxidase, GABA oxidase, histamine oxidase, diamine oxidase, nucleoside oxidase, L-lysine oxidase, L-aspartate oxidase, glycine oxidase, galactose oxidase.

89. The assembly of claim 77, wherein said layer comprises a plurality of co-factors for said enzyme.

90. The assembly of claim 77, wherein said enzyme is crosslinked with said matrix.

91. The assembly of claim 77, wherein said enzyme is entrapped in said matrix.

92. The assembly of claim 77, wherein said enzyme is polymerized with said matrix.

93. The assembly of claim 77, wherein said matrix is selected from the group consisting of low molecular weight bis-aldehydes, glutaraldehyde and serum albumen, polyphenols, polypyrroles, polyphenylenediamines, polyurethanes, sulfonated polytetrafluoroethylenes, cellulose acetates, sol gels, and zeolites.

94. The assembly of claim 77, wherein said co-factor is selected from the group consisting of FAD, FMN, thiamine pyrophosphate, quinone, home, porphorin, and heavy metals.

95. The assembly of claim 77, wherein said support comprises a body selected from the group consisting of cylindrical bodies and planar bodies.

96. The assembly of claim 77, said matrix retaining said enzyme in said active form for at least about 24 hours.

97. The assembly of claim 96, said matrix retaining said enzyme in said active form for at least about 48 hours.

98. The assembly of claim 97, said matrix retaining said enzyme in said active form for at least about 72 hours.

99. The assembly of claim 98, said matrix retaining said enzyme in said active form for at least about 1 week.

100. The assembly of claim 99, said matrix retaining said enzyme in said active form for at least about 2 weeks.

101. The assembly of claim 100, said matrix retaining said enzyme in said active form for at least about 3 weeks.

102. The assembly of claim 101, said matrix retaining said enzyme in said active form for at least about 4 weeks.

103. The assembly of claim 102, said matrix retaining said enzyme in said active form for at least about 8 weeks.

104. The assembly of claim 103, said matrix retaining said enzyme in said active form for at least about 12 weeks.

105. The assembly of claim 104, said matrix retaining said enzyme in said active form for at least about 16 weeks.

106. The assembly of claim 105, said matrix retaining said enzyme in said active form for at least about 30 weeks.

107. The assembly of claim 106, said matrix retaining said enzyme in said active form for at least about 52 weeks.
